(19) United States
(12) Patent Application Publication
(10) Pub. No.: US 2024/0293791 A1
(43) Pub. Date: Sep. 5, 2024

(54) COLOR-NEUTRAL DEGRADABLE MICROCAPSULES

(71) Applicant: KOEHLER INNOVATION & TECHNOLOGY GMBH, Oberkirch (DE)

(72) Inventors: Claudia MEIER, Lichtenau (DE); Jeanette HILDEBRAND, Buhl (DE); Michael BIEDENBACH, Oberkirch (DE); Christian KIND, Baden-Baden (DE)

(73) Assignee: KOEHLER INNOVATION & TECHNOLOGY GMBH, Oberkirch (DE)

(21) Appl. No.: 18/568,552

(22) PCT Filed: Jun. 10, 2022
(86) PCT No.: PCT/DE2022/200122

§ 371 Date: Dec. 8, 2023

(30) Foreign Application Priority Data
Jun. 11, 2021 (DE) 10 2021 205 957.0

(51) Int. Cl. B01J 13/18; A61K 9/50; B01J 13/22; C11B 9/00

(52) U.S. Cl. CPC B01J 13/18; A61K 9/5026; B01J 13/22; C11B 9/0003; C09J 2425/00; C09J 2433/00

(57) ABSTRACT

According to a first aspect, the invention relates to biodegradable microcapsules, comprising a core material and a shell, wherein the shell consists of at least one barrier layer and at least one stability layer, wherein the barrier layer surrounds the core material, wherein the stability layer comprises at least one biopolymer, and is arranged on the outer surface of the barrier layer, and wherein an emulsion stabilizer is arranged at the transition from barrier layer to stability layer. Furthermore, the invention relates to the use of an emulsion stabilizer to increase the amount of a stability layer that can be deposited on the surface of a barrier layer, a product containing the biodegradable microcapsules, and a method for producing the biodegradable microcapsules.

### FIELD OF THE INVENTION

The invention relates to improved biodegradable microcapsules with environmentally compatible wall materials for use in applications with high demands on the impermeability and stability of the microcapsules, as well as their manufacturing process. The invention further relates to microcapsule dispersions consisting of these microcapsules with a specific coloration.

### BACKGROUND OF THE INVENTION

Microencapsulation is a versatile technology. It offers solutions for numerous innovations—from the paper industry to household products, microencapsulation increases the functionality of a wide range of active substances. Encapsulated active substances can be used more economically and improve the sustainability and environmental compatibility of many products

However, the polymeric wall materials of the microcapsules themselves are environmentally compatible to very different degrees. Microcapsule walls based on the natural product gelatin and thus completely biodegradable have long been used in carbonless paper. A gelatin encapsulation process developed as early as the 1950s is disclosed in U.S. Pat. No. 2,800,457. Since then, a variety of variations in terms of materials and process steps have been described. In addition, biodegradable or enzymatically degradable microcapsule walls are used to employ enzymatic degradation as a method for releasing the core material. Such microcapsules are described, for example, in WO 2009/126742 A1 or WO 2015/014628 A1.

However, such microcapsules are not suitable for many industrial applications and household products. This is because natural-substance-based microcapsules do not meet the diffusion impermeability, chemical resistance and temperature resistance required for detergents and cleaning agents, adhesive systems, coatings and dispersions, for example, nor the required loading with core material.

In these so-called high-demand areas, classically organic polymers such as melamine-formaldehyde polymers (see e.g., EP 2 689 835 A1, WO 2018/114056 A1, WO 2014/016395 A1, WO 2011/075425 A1 or WO 2011/120772 A1); Polyacrylates (see e.g., WO 2014/032920 A1, WO 2010/79466 A2); Polyamides; Polyurethane or polyureas (see e.g., WO 2014/036082 A2 or WO 2017/143174 A1). The capsules constructed of such organic polymers have the required diffusion impermeability, stability and chemical resistance. However, these organic polymers are only enzymatically or biodegradable to a very limited extent.

In the current state of the art, various approaches are described in which biopolymers are combined as an additional component with the organic polymers of the microcapsule shell for use in high-demand areas. However, this is not with the goal of producing biodegradable microcapsules, but primarily to alter the release, stability, or surface properties of the microcapsules. For example, in WO 2014/044840 A1, a method for producing two-layered microcapsules described with an inner polyurea layer and an outer gelatin-containing layer. The polyurea layer is generated by polyaddition on the inside of the gelatin layer obtaining by coacervation. The capsules thus obtained have the necessary stability and impermeability for use in detergents and cleaning agents due to the polyurea layer and additionally by the gelatin to attach them to surfaces. Concrete stabilities and resistance are not mentioned. A disadvantage of poly urea capsules, however, is the inevitable side reaction of the core materials having the diisocyanates used to produce the urea, which must be added to the oil-based core.

On the other hand, biopolymer-based microcapsules are also described in the prior art, which achieve improved impermeability or stability against environmental influences or a targeted setting of a delayed release behavior by adding a protective layer. For example, WO 2010/003762 A1 describes particles with a core-shell-shell structure. Inside each particle there is a poorly water-soluble or water-insoluble organic active ingredient as a core. The shell directly surrounding the core contains a biodegradable polymer and the outer shell contains at least one metal or semimetal oxide. With this structure, a biodegradable shell is indeed obtained. Nevertheless, the microcapsules are used in food, cosmetics or pharmaceuticals according to WO 2010/003762 A1, but are not usable for the high demand areas according to the invention due to lack of impermeability.

The applicant has described in the unpublished PCT/EP2020/085804 microcapsules with a multi-layered structure of the shells, which are essentially biodegradable yet possess sufficient stability and impermeability to be used in high-demand areas such as detergents and cleaning agents. This is achieved by a stability layer forming the main part of the capsule shell, made from naturally occurring and highly biodegradable materials, such as gelatin or alginate, or from materials ubiquitously present in nature.

This stability layer is combined with a barrier layer, which may consist of known materials used in microencapsulation, such as melamine-formaldehyde or meth(acrylate). It has been possible to design the barrier layer with a previously unachievable low wall thickness while still ensuring sufficient impermeability. As a result, the proportion of the barrier layer in the total wall is kept very low, so that the microcapsule wall exhibits a biodegradability measured according to OECD 301 F of at least 40%.

### SUMMARY OF THE INVENTION

The present invention is based, inter alia, on the discovery that microcapsules with a multi-layer structure of the shell, consisting of a highly biodegradable stability layer and a thin barrier layer, can be improved by using emulsion stabilizers after the production of the inner barrier layer. Emulsion stabilizers are regularly used to stabilize the core material emulsion. Surprisingly, however, it has been shown that treating the surface of the barrier layer enveloping the core material with an emulsion stabilizer, especially a copolymer containing certain acrylic acid derivatives, leads to improved deposition of the stability layer and thus to a greater average layer thickness of the stability layer (see Examples 2 to 4).

Consequently, the invention, according to a first aspect, concerns biodegradable microcapsules, comprising a core material and a shell, wherein the shell consists of at least a barrier layer and a stability layer, wherein the barrier layer surrounds the core material, wherein the stability layer comprises at least one biopolymer, and is arranged on the outer surface of the barrier layer, and wherein an emulsion stabilizer is arranged at the transition from the barrier layer to the stability layer.

The emulsion stabilizer according to the invention is a polymer or copolymer, constructed from certain acrylic acid derivatives, N-vinylpyrrolidone; and/or styrene.

Thus, according to a second aspect, the invention concerns the use of an emulsion stabilizer for increasing the amount of a stability layer that can be deposited on the surface of a barrier layer, wherein the barrier layer and the stability layer form the capsule wall of a microcapsule, wherein the emulsion stabilizer is preferably a polymer or copolymer, consisting of one or more monomers selected from:
(1) Acrylic acid derivatives of the general formula (I)

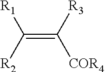

wherein
R₁, R₂ and R₅ are selected from hydrogen and a C₁₋₄-alkyl group, wherein R₁ and R₂ are particularly hydrogen and R₃ is particularly hydrogen or methyl; and R₄ is for —OX or —NR₅R₆, wherein X is hydrogen, an alkali metal, an ammonium group, or a C₁-C₁₈ alkyl possibly substituted by —SO₃M or —OH, wherein M is hydrogen, an alkali metal, or ammonium, wherein the optionally-SO₃M or —OH substituted C₁-C₁₈ alkyl is preferably methyl, ethyl, n-butyl, 2-ethylhexyl, 2-sulfoethyl, or 3-sulfopropyl, wherein R₅ and R₆ are independently selected from hydrogen or a C₁-C₁₀ alkyl possibly substituted by —SO₃M, wherein at least one of R₅ and R₆ is not hydrogen, and wherein preferably R₅ is H and R₆ is 2-methyl-propan-2-yl-1-sulfonic acid;
(2) N-vinylpyrrolidone; and
(3) styrene.

The emulsion stabilizer is preferably an acrylate copolymer containing 2-acrylamido-2-methylpropanesulfonic acid (AMPS). A suitable copolymer is available, for example, under the trade name Dimension PA 140.

In various embodiments of the second aspect, the barrier layer is composed of one or more components selected from the group consisting of an aldehydic component, an aromatic alcohol, an amine component, an acrylate component, and an isocyanate component, and the stability layer comprises at least one biopolymer.

Furthermore, it is advantageous that the improved structural integration of the stability layer by the barrier layer, through the addition of the emulsion stabilizer, ensures the structural (covalent) connection of all wall-forming components, so that the individual layers are inseparably connected and can be considered as a monopolymer.

Due to the robustness and impermeability of the biodegradable capsule, it can be used in a variety of products. Consequently, according to a third aspect, the invention concerns a product comprising microcapsules according to the first aspect, wherein the product is selected from the group consisting of an adhesive system; a pharmaceutical product; a coating material, particularly a coated paper; a thermal storage coating, a self-healing coating, or a corrosion protection coating; and coatings containing such microcapsules for functional packaging materials.

Furthermore, the invention, according to a fourth aspect, concerns the use of microcapsules according to the first aspect for the production of a product according to the third aspect.

According to a fifth aspect, the invention concerns a method for producing biodegradable microcapsules according to the first aspect, wherein the method comprises the following steps:
a) preparing an oil-in-water emulsion by emulsifying a core material in an aqueous phase in the presence of the wall-forming component(s) of the inner barrier layer with the addition of protective colloids;
b) deposition and curing of the wall-forming component(s) of the barrier layer, wherein the wall-forming component(s) of the barrier layer preferably are an aldehydic component, an amine component, and an aromatic alcohol, particularly preferably formaldehyde, melamine, and resorcin;
c) addition of an emulsion stabilizer and
d) addition of the wall-forming component(s) of the stability layer, followed by deposition and curing, wherein the wall-forming component(s) of the stability layer are at least one biopolymer, preferably a protein and/or a polysaccharide, particularly preferably gelatin and alginate, as well as a curing agent, preferably glutaraldehyde or glyoxal; and
e) optionally, the addition of the wall-forming component(s) of the outer, third shell layer, followed by deposition and curing, wherein the wall-forming component(s) of the outer, third shell layer preferably comprise an amine component, in particular melamine.

Another advantage of the microcapsules obtained with the manufacturing process described herein is the light color of the microcapsule dispersions. Thus, according to a sixth aspect, the invention concerns a microcapsule dispersion containing biodegradable microcapsules according to the first aspect, wherein said microcapsules have a color coordinate in the L*a*b* color space with an L* value of at least 50.

### FIGURES

FIG. 1 shows a light microscopic image of various microcapsule dispersions, each on the left at 50× and 500× magnification taken with an Olympus BX 50 microscope.
A) Microcapsule dispersion according to the invention Slurry 2
B) Microcapsule dispersion according to the invention Slurry 3
C) Microcapsule dispersion according to the invention Slurry 5
D) Microcapsule dispersion according to the invention Slurry 6
E) Reference microcapsule dispersion MK1
F) Reference microcapsule dispersion MK2
G) Reference microcapsule dispersion MK4

FIG. 2 shows two microscopic images of the microcapsule dispersions Slurry 2 and MK1 with guide lines for determining the thickness of the stability layer of the microcapsules and information on the measured thicknesses.

FIG. 3 shows a chart of the course of biological degradation according to OECD 301 F over 60 days after washing of the microcapsule dispersions according to the invention and references. Slurry 2 and Slurry 3 as well as the reference microcapsule dispersion MK1. In A) the degradation of Slurry 2 and Slurry 3 is shown, along with the degradation of ethylene glycol and walnut shell flour as positive controls. In B) Slurry 2 and Slurry 3 are compared with the reference capsule MK1.

FIG. 4 shows a chart of the storage stability of various microcapsule dispersions as described in Example 5. A) Stability of the microcapsule dispersions Slurry 2 and Slurry 3 during storage over a period of 4 weeks compared with the reference capsule MK2. B) Stability of the microcapsule dispersion Slurry 2 during storage over a period of 4 weeks compared with the reference capsules MK1 and MK2. C) Stability of the microcapsule dispersion Slurry 3 during storage over a period of 4 weeks compared with the reference capsules MK2 and MK4. D) Stability of the microcapsule dispersion Slurry 5 and Slurry 6 compared with the reference capsule MK2.

FIG. 5 shows the chart of the development of the L*a*b* values of the microcapsule dispersions Slurry 3 and 3A over a period of 8 days. A) Temporal development of the L* value. B) Temporal development of the a* value. C) Temporal development of the b* value.

### DETAILED DESCRIPTION OF THE INVENTION

### Definitions

“Barrier layer” refers to the layer of a microcapsule wall that is essentially responsible for the impermeability of the capsule shell, i.e., it prevents the release of the core material.

“Biodegradability” refers to the ability of organic chemicals to be biologically decomposed, that is, by living organisms or their enzymes. Ideally, this chemical metabolism proceeds completely to mineralization, but may stop at degradation-stable transformation products. Generally accepted are the guidelines for testing chemicals of the OECD, which are also used in the context of chemical approval. The tests of the OECD Test Series 301 (A-F) demonstrate rapid and complete biodegradation (ready biodegradability) under aerobic conditions. Different test methods are available for well or poorly soluble as well as for volatile substances. In particular, the manometric respiration test (OECD 301 F) is used within the scope of the application. The basic biodegradability (inherent biodegradability) can be determined via the measurement standard OECD 302, for example the MITI I I test (OECD 302 C).

“Biodegradable” or “biologically degradable” in the sense of the present invention refers to microcapsule walls that exhibit a biodegradability, measured according to OECD 301 F, of at least 40% within 60 days. Microcapsule walls that show a degradation of at least 60% within 60 days, measured according to OECD 301 F, are also referred to here as rapidly biodegradable.

A “biopolymer” is a naturally occurring polymer, for example, a polymer found in a plant, fungus, bacterium, or animal. Biopolymers also include modified polymers based on naturally occurring polymers. The biopolymer can be derived from the natural source or synthetically produced.

“Impermeability” to a substance, gas, liquid, radiation or the like, is a property of material structures. According to the invention, the terms “impermeability” and “tightness” are used synonymously. Impermeability is a relative term and always refers to given general conditions.

“Emulsion stabilizers” are auxiliaries used for stabilizing emulsions. The emulsion stabilizers can be added in small amounts to the aqueous or oily phase (of emulsions) where they become enriched at the interface in a phase-oriented manner, thereby facilitating the dispersion of the internal phase by reducing the interfacial tension and increasing the resistance to dispersion of the emulsion.

“High demand areas” in the sense of the invention are application areas with high demands on the impermeability and stability of the microcapsules.

The term “(meth)acrylate” in the present invention refers to both methacrylates and acrylates.

According to the invention, the term “microcapsules” refers to particles containing an inner space or core filled with a solid, gelled, liquid or gaseous medium and enclosed (encapsulated) by a continuous coating (shell) of film-forming polymers. These particles preferably have small dimensions. The terms “microcapsules,” “core-shell capsules”, or simply “capsules” are used interchangeably.

“Microencapsulation” refers to a manufacturing process in which small and miniscule portions of solid, liquid or gaseous substances are surrounded by a shell of polymeric or inorganic wall materials. The microcapsules thus obtained may have a diameter of a few millimeters to less than 1 μm.

The microcapsule according to the invention features a multi-layered “shell.” The shell enclosing the core material of the microcapsule is also regularly referred to as the “wall” or “sheath.

The microcapsules according to the invention have a shell consisting of several components with different functions. These components are gradually reacted and covalently bonded as a whole through aldehydic crosslinking. In the context of describing the capsule structure, the components are referred to as individual layers or individual shells. Thus, “multi-layered” and “multi-shelled” are used synonymously.

“Stability layer” refers to the layer of a capsule wall that is essentially responsible for the stability of the capsule shell, i.e., usually constitutes the main part of the shell.

“Wall formers” are the components that build up the microcapsule wall.

### Microcapsules

According to a first aspect, the invention concerns biodegradable microcapsules, comprising a core material and a shell, wherein the shell consists of at least a barrier layer and a stability layer, wherein the barrier layer surrounds the core material, wherein the stability layer comprises at least one biopolymer, and is arranged on the outer surface of the barrier layer, and wherein an emulsion stabilizer is arranged at the transition from the barrier layer to the stability layer. This arrangement can consist of an intermediate layer of emulsion stabilizer, which can be continuous or discontinuous and can cover parts or the entire inner barrier layer. Alternatively, individual molecules of the emulsion stabilizer can be arranged on the surface of the barrier layer in such a way that they facilitate a bond between the stability layer and the barrier layer. The emulsion stabilizer acts here as a linking agent

As shown in Example 4, the microcapsule shells according to the invention, due to the application of the emulsion stabilizer, exhibit a significantly increased thickness of the stability layer. As a result, the proportion of natural components in the capsule is further increased compared to the previously described multi-layer microcapsules.

According to one embodiment of the biodegradable microcapsules, during the production of the microcapsules, the surface of the barrier layer is brought into contact with the emulsion stabilizer before the formation of the stability layer. This increases the capacity of the surface for structural attachment of the stability layer. Without wanting to be bound by theory, the inventors assume that the emulsion stabilizer attaches to the partially polar surface of the barrier layer, in particular a melamine-resorcin-formaldehyde layer, thus providing both a framework to the biopolymers of the stability layer and the sufficient non-polar surface characteristics for the deposition of the formed coacervate. This not only increases the average layer thickness of the stability layer produced with the biopolymer but also incorporates the emulsion stabilizer at the interface between the stability layer and the barrier layer. Based on this theory, essentially any emulsion stabilizer could act as a linking agent for the production of the microcapsules according to the invention.

In a preferred embodiment, the emulsion stabilizer is a polymer or copolymer, consisting of one or more monomers selected from:
(1) Acrylic acid derivatives of the general formula (I)

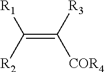

wherein
R₁, R₂ and R₅ are selected from hydrogen and a C₁₋₄ alkyl group; and R₄ stands for —OX or —NR₅R₆, where X is hydrogen, an alkali metal, an ammonium group, or a C₁-C₁₈ alkyl possibly substituted by —SO₃M or —OH, where M is hydrogen, an alkali metal, or ammonium, where R₅ and R₆ independently stand for hydrogen or a C₁-C₁₀ alkyl possibly substituted by —SO₃M, with at least one of R₅ and R₆ being not hydrogen,
(2) N-vinylpyrrolidone; and
(3) styrene.

The possible C₁₋₄ hydroxyalkyl groups for R₁, R₂, and R₃ can be ethyl, n-propyl, i-propyl, and n-butyl. In some embodiments of the acrylic acid derivatives, R₁ and R₂ are hydrogen, and R₃ is hydrogen or methyl. Depending on the selection of R₃, it is an acrylate (hydrogen) or methacrylate (methyl).

The possible C₁-C₁₈ alkyl groups for X, which may be substituted by —OH or —SO₃M, are preferably selected from methyl, ethyl, C₂₋₄ hydroxyalkyl, C₂₋₄ sulfoalkyl, and C₄-C₁₈ alkyl groups.

The C₂₋₄ hydroxyalkyl groups can be selected from ethyl, n-propyl, i-propyl, and n-butyl. As unsubstituted C₄₋₁₈ alkyl groups, examples include n-butyl, sec-butyl, tert-butyl, pentyl, hexyl, ethylhexyl, octyl, decyl, dodecyl, or stearyl groups. Particularly suitable are n-butyl and ethylhexyl. Ethylhexyl is particularly 2-ethylhexyl. As C₂₋₄ sulfoalkyl groups, particularly 2-sulfoethyl and 3-sulfopropyl are noteworthy.

According to one embodiment of the acrylic acid derivatives, R₄ is-NR₅R₆, where R₅ is H and R₆ is 2-methylpropan-2-yl-1-sulfonic acid. Particularly in this case, R₁, R₂, and R₃ are hydrogen.

According to one embodiment, R₄ is-OX and X is hydrogen. Particularly, R₁, R₂, and R₃ are hydrogen (acrylic acid). Alternatively, R₃ is methyl (methacrylate). According to an embodiment of the acrylic acid derivatives, R₄ is-OX and X is methyl. Particularly, R₁, R₂, and R₃ are hydrogen (methyl acrylate). According to one embodiment, R₄ is-OX and X is 2-ethylhexyl. Particularly, R₁, R₂, and R₃ are hydrogen (ethylhexyl acrylate). According to one embodiment, R₄ is-OX and X is n-butyl. Particularly, R₁, R₂, and R₅ are hydrogen (n-butyl acrylate). According to one embodiment of the acrylic acid derivatives, R₄ is-OX and X is 2-sulfoethyl. Particularly, R₁, R₂, and R₃ are hydrogen (sulfoethyl acrylate). According to one embodiment of the acrylic acid derivatives, R₄ is-OX and X is 3-sulfopropyl. Particularly, R₁, and R₂ are hydrogen and R₃ is methyl (sulfopropyl(meth)acrylate).

The polymers or copolymers composed of monomers of formula (I) typically conform to formula (II):

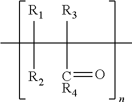

wherein R1-R4 have the meanings given above and n is a whole number of at least 3. For example, n may be greater than 5, 10, 20, 30, 40, 50, 60, 70, 80, or 100. n may be, for example, less than 10,000, 7,500, 5,000, 2,500, 1,000, or 500. According to one embodiment, n is in the range of 5 to 5,000. In one embodiment, n is in the range of 10 to 1,000

This group of polymers and copolymers represents a sensible generalization of the copolymers present in Dimension PA 140. The emulsion stabilizer is preferably an acrylate copolymer containing at least two different monomers of formula (I). In various embodiments, the copolymer contains AMPS, optionally in combination with (meth)acrylic acid and/or at least one alkyl (meth)acrylate. According to one embodiment, the copolymer contains AMPS and one or more monomers selected from acrylate, methacrylate, methyl acrylate, ethylhexyl acrylate, n-butyl acrylate, N-vinylpyrrolidone, and styrene.

According to one embodiment, the copolymer contains AMPS, acrylate, methyl acrylate, and styrene. According to another embodiment, the copolymer contains AMPS, acrylate, methyl acrylate, and ethylhexyl acrylate. According to one embodiment, the copolymer contains AMPS, methyl acrylate, N-vinylpyrrolidone, and styrene. According to one embodiment, the copolymer contains AMPS, acrylate, methyl acrylate, and ethylhexyl acrylate. According to one embodiment, the copolymer contains AMPS, methyl acrylate, N-vinylpyrrolidone, and styrene. According to one embodiment, the copolymer contains AMPS, methyl acrylate, and styrene. According to one embodiment, the copolymer contains AMPS, methacrylate, and styrene. According to one embodiment, the copolymer contains AMPS, acrylate, methyl acrylate, and n-butyl acrylate.

According to one embodiment, the emulsion stabilizer is a copolymer as defined in EP0562344B1, which is incorporated herein by reference. According to one embodiment, the emulsion stabilizer is a copolymer containing a) AMPS, sulfoethyl or sulfopropyl (meth)acrylate, or vinyl sulfonic acid, especially with a proportion of 20 to 90%; b) a vinyl unsaturated acid, especially with a proportion of 0 to 50%; c) methyl or ethyl acrylate or methacrylate, C₂₋₄ hydroxyalkyl acrylate, or N-vinylpyrrolidone, particularly with a proportion of 0 to 70%; and d) styrene or C₄₋₁₈ alkyl acrylate or C₄₋₁₈ alkyl methacrylate, particularly with a proportion of 0.1 to 10%.

According to one embodiment, the emulsion stabilizer is a copolymer containing a) 2-acrylamido-2-methylpropane sulfonic acid, sulfoethyl or sulfopropyl (meth)acrylate, or vinyl sulfonic acid, particularly with a proportion of 40 to 75% b) acrylic acid or methacrylic acid, particularly with a proportion of 10 to 40% c) methyl or ethyl acrylate or methacrylate, C₂₋₄ hydroxyalkyl acrylate, or N-vinylpyrrolidone, particularly with a proportion of 10 to 50% and d) 0.5 to 5% styrene or C4-18 alkyl acrylate or methacrylate, particularly with a proportion of 0.5 to 5%.

According to one embodiment, the emulsion stabilizer is a copolymer containing a) 40 to 75%2-acrylamido-2-methylpropane sulfonic acid, sulfoethyl or sulfopropyl (meth)acrylate, or vinyl sulfonic acid, particularly with a proportion of 40 to 75% b) acrylic acid or methacrylic acid, 10 to 30% c) methyl or ethyl acrylate or methacrylate, or N-vinylpyrrolidone, particularly with a proportion of 10 to 50% and d) styrene or C4-18 alkyl acrylate or methacrylate, particularly with a proportion of 0.5 to 5%.

A suitable copolymer is available, for example, under the trade name Dimension PA 140 (Solenis).

According to one embodiment, the emulsion stabilizer does not consist of or comprise N-vinylpyrrolidone, polyvinylpyrrolidone homopolymer, or polyvinylpyrrolidone copolymer.

The precise determination of the proportion of the emulsion stabilizer in the stabilization layer is technically challenging. However, unlike its use as a protective colloid in the encapsulation of the core material, it is assumed that a significant part of the emulsion stabilizer is incorporated into the microcapsule shell

The proportion of the emulsion stabilizer used in the components for microencapsulation can range from 0.1 to 15 wt. %. For example, the proportion of the emulsion stabilizer used can be 0.1 wt. %, 0.2 wt. %, 0.5 wt. %, 1 wt. %, 2 wt. %, 3 wt. %, 4 wt. %, 5 wt. %, 6 wt. %, 7 wt. %, 8 wt. %, 9 wt. %, 10 wt. %, 11 wt. %, 12 wt. %, 13 wt. %, 14 wt. %, or 15 wt. %. Below a concentration of 0.1 wt. %, there is a risk that the surface of the barrier layer is not sufficiently covered with the emulsion stabilizer to ensure the inventive effect, namely the increase in the deposition amount of the stability layer. Above 15 wt. %, the high concentration of an emulsion stabilizer can hinder the formation of the stability layer. In a preferred embodiment, the emulsion stabilizer is used with a proportion of the components used for microencapsulation in the range of 0.25 wt. % to 5 wt. %. In a particularly preferred embodiment, the proportion of the emulsion stabilizer used is in the range of 0.5 wt. % to 4 wt. %.

Assuming that at least part of the emulsion stabilizer is incorporated into the microcapsule wall, according to one embodiment, the proportion of the emulsion stabilizer relative to the total weight of the microcapsule wall is in the range of 0.5 to 15.0 wt. %. For example, the proportion of the emulsion stabilizer used can be 0.5 wt. %, 1.0 wt. %, 1.5 wt. %, 2.0 wt. %, 2.5 wt. %, 3 wt. %, 4 wt. %, 5 wt. %, 6 wt. %, 7 wt. %, 8 wt. %, 9 wt. %, 10 wt. %, 11 wt. %, 12 wt. %, 13 wt. %, 14 wt. %, or 15 wt. %. In a preferred embodiment, the proportion of the wall-forming components of the microcapsule shell is in the range of 1 wt. % to 11 wt. %. In a particularly preferred embodiment, the proportion of the emulsion stabilizer used is in the range of 2 wt. % to 7 wt. %.

The barrier layer preferably contains, as wall formers, one or more components selected from the group consisting of an aldehydic component, an aromatic alcohol, an amine component, and an acrylate component. Manufacturing methods for producing microcapsules with these wall materials are known the skilled person. For the production of the barrier layer, a polymer selected from a polycondensation product of an aldehydic component with one or more aromatic alcohols, and/or amine components can be used.

As shown in Examples 2 and 3, the thin wall thickness of the barrier layer can particularly be achieved with a melamine-formaldehyde layer containing aromatic alcohols or m-aminophenol. Consequently, the barrier layer preferably includes an aldehydic component, an amine component, and an aromatic alcohol.

The use of amine-aldehyde compounds in the barrier layer, particularly melamine-formaldehyde, has the advantage that these compounds form a hydrophilic surface with a high proportion of hydroxy functionality, thereby showing basic compatibility with the coacervation partners of the stability layer, such as biodegradable proteins, polysaccharides, chitosan, lignins, and phosphazenes, as well as inorganic wall materials such as CaCO3 and polysiloxanes. Similarly, polyacrylates, particularly from components such as styrene, vinyl compounds, methyl methacrylate, and 1,4-butanediol acrylate, methacrylic acid, can be generated as a microcapsule wall by initiation, for example, with t-butyl hydroperoxide in a radically induced polymerization (polyacrylates), forming a hydrophilic surface with a high proportion of hydroxy functionality, and therefore are equally compatible with the components according to the invention of the stability layer.

In a preferred embodiment, thus, a wall former of the barrier layer is an aldehydic component. According to one embodiment, the aldehydic component of the barrier layer is selected from the group consisting of formaldehyde, glutaraldehyde, succinaldehyde, furfural, and glyoxal. Microcapsules have been successfully produced with all these aldehydes (see WO 2013 037 575 A1), so it can be assumed that similarly impermeable capsules as with formaldehyde can be obtained.

Based on the examples, the proportion of the aldehydic component for wall formation relative to the total weight of the barrier layer should be in the range of 5 wt. % to 50 wt. %. For example, the proportion of the aldehydic component can be 5 wt. %, 6 wt. %, 7 wt. %, 8 wt. %, 9 wt. %, 10 wt. %, or 15 wt. %, 20 wt. %, 25 wt. %, 30 wt. %, 35 wt. %, 40 wt. %, 45 wt. %, or 50 wt. %. It is assumed that outside these limits, no sufficiently stable and dense, thin layer can be obtained. Preferably, the concentration of the aldehydic component in the barrier layer is in the range of 10 wt. % to 30 wt. %. Particularly preferred is the concentration of the aldehydic component in the barrier layer in the range of 15 wt. % to 20 wt. %.

As amine component in the barrier layer, particularly melamine, melamine derivatives, and urea, or combinations thereof are suitable. Appropriate melamine derivatives are etherified melamine derivatives as well as methylolated melamine derivatives. Melamine in the methylolated form is preferred. The amine components can, for example, be used in the form of alkylated mono- and polymethylol urea precondensation products or partially methylolated mono- and polymethylol-1,3,5-triamino-2,4,6-triazine precondensation products like Dimension SD® (by Solenis). According to one embodiment, the amine component is melamine. According to an alternative embodiment, the amine component is a combination of melamine and urea.

The aldehydic component and the amine component can be present in a molar ratio in the range of 1:5 to 3:1. For example, the molar ratio can be 1:5, 1:4.5, 1:4, 1:3.5, 1:3, 1:2.5, 1:2, 1:1.8, 1:1.6, 1:1.4, 1:1.35, 1:1.3, 1:1.2, 1:1, 1.5:1, 2:1, 2.5:1, or 3:1. Preferably, the molar ratio is in the range of 1:3 to 2:1. Particularly preferred, the molar ratio of the aldehydic component and the amine component can be in the range of 1:2 to 1:1. The aldehydic component and the amine component are typically used in a ratio of about 1:1.35. This molar ratio allows a complete reaction of the two reactants and leads to high impermeability of the capsules. Aldehyde-amine capsule walls with a molar ratio of 1:2 are also known, for example. These capsules have the advantage that the proportion of the highly crosslinking aldehyde, particularly formaldehyde, is very low. However, these capsules have a lower impermeability than those with a ratio of 1:1.35. Capsules with a ratio of 2:1 have increased impermeability but have the disadvantage that the aldehyde component is partially unreacted in the capsule wall and the slurry.

According to one embodiment, the proportion of the amine component(s) (e.g., melamine and/or urea) in the barrier layer, relative to the total weight of the barrier layer, is in the range of 20 wt. % to 85 wt. %. For example, the proportion of the amine component can be 20 wt. %, 25 wt. %, 30 wt. %, 35 wt. %, 40 wt. %, 45 wt. %, 50 wt. %, 55 wt. %, 60 wt. %, 65 wt. %, 70 wt. %, 75 wt. %, 80 wt. % or 85 wt. %. In a preferred embodiment, the proportion of the amine component in the barrier layer, relative to the total weight of the barrier layer, is in the range of 40 wt. % to 80 wt. %. Particularly preferred is the proportion of the amine component in the range of 55 to 70 wt. %.

With the aromatic alcohol, it is possible to greatly reduce the wall thickness of the barrier layer constructed from the amine component and the aldehyde component, while still obtaining a layer that has the necessary impermeability and is at least stable enough in combination with the stability layer. The aromatic alcohols impart increased impermeability to the wall, as their strongly hydrophobic aromatic structure makes it more difficult for low molecular weight substances to diffuse through. As illustrated in the examples, phloroglucinol, resorcin, or m-aminophenol are particularly suitable as aromatic alcohols. Consequently, in one embodiment, the aromatic alcohol is selected from the group consisting of phloroglucinol, resorcin, and aminophenol. In combination with the amine and aldehyde components, the aromatic alcohol is used in a molar ratio to the aldehyde component in the range of (alcohol:aldehyde) 1:1 to 1:20, preferably in the range of 1:2 to 1:10.

According to one embodiment, the proportion of the aromatic alcohol in the barrier layer, relative to the total weight of the barrier layer, is in the range of 1.0 wt. % to 20 wt. %. For example, the proportion of the aromatic alcohol can be 1.5 wt. %, 2.0 wt. %, 2.5 wt. %, 3.0 wt. %, 4.0 wt. %, 5.0 wt. %, 6 wt. %, 7 wt. %, 8 wt. %, 9 wt. %, 10 wt. %, 11 wt. %, 12 wt. %, 13 wt. %, 14 wt. %, 15 wt. %, 16 wt. %, 17 wt. %, 18 wt. %, 19 wt. % or 20 wt. %. Due to their aromatic structure, the aromatic alcohols give the capsule wall a coloration that increases with the proportion of the aromatic alcohol. Such coloring is undesirable in a variety of applications. Moreover, the aromatic alcohols are prone to oxidation, which leads to a change in color over time. Therefore, it can be difficult to balance the undesirable coloration of the microcapsules with a dye. Thus, the aromatic alcohols should not be used above 20.0 wt. %. Below 1.0 wt. %, no effect on impermeability is detectable. In a preferred embodiment, the proportion of the aromatic alcohol in the barrier layer, relative to the total weight of the barrier layer, is in the range of 5.0 wt. % to 15.0 wt. %. Up to a percentage of 15.0 wt. %, the coloring is tolerable in most applications. In a particularly preferred embodiment, the proportion of the aromatic alcohol in the barrier layer, relative to the total weight of the barrier layer, is in the range of 6 wt. % to 16.0 wt. %. Particularly, the proportion of the aromatic alcohol in the barrier layer is in the range of 10 wt. % to 14.0 wt. %.

According to another embodiment, the aldehyde component of the barrier layer can be used together with an aromatic alcohol such as resorcin, phloroglucinol, or m-aminophenol as wall-forming component(s), i.e., without the amine component(s).

According to one embodiment, the barrier layer contains melamine, formaldehyde, and resorcin. In another embodiment, the barrier layer of the microcapsules contains melamine, urea, formaldehyde, and resorcin. In a preferred embodiment, the barrier layer contains melamine in the range of 25 to 40 wt. %, formaldehyde in the range of 15 to 20 wt. %, and resorcin in the range of 10 to 14 wt. %, and optionally urea in the range of 25 to 35 wt. %. The proportions refer to the amounts used for wall formation of the layer and are relative to the total weight of the barrier layer without protective colloid.

For encapsulating the core material with the barrier layer consisting of an aldehydic component, an amine component, and an aromatic alcohol, an emulsion stabilizer is preferably used as a protective colloid, as mentioned above. The emulsion stabilizer used as a protective colloid can be a polymer or copolymer as defined above as a linking agent. For example, the protective colloid is a copolymer (AMPS, Dimension® PA 140, Solenis) or its salts. In one embodiment, the same copolymer is used as a protective colloid and as a linking agent.

As amine component in the barrier layer, particularly melamine, melamine derivatives, and urea, or combinations thereof are suitable. Suitable melamine derivatives are etherified melamine derivatives and methylolated melamine derivatives. Melamine in the methylolated form is preferred. The amine components can, for example, be used in the form of alkylated mono- and polymethylol urea precondensation products or partially methylolated mono- and polymethylol-1,3,5-triamino-2,4,6-triazine precondensation products such as Dimension SD® (from Solenis). According to one embodiment, the amine component is melamine. According to an alternative embodiment, the amine component is a combination of melamine and urea.

The stability layer forms the main component of the microcapsule shell and thus ensures a high biodegradability according to OECD 301 F of at least 40% within 60 days. Suitable biopolymers as wall formers for the stability layer are proteins such as gelatin, whey protein, plant storage protein; polysaccharides such as alginate, gum arabic, modified gum, chitin, dextran, dextrin, pectin, cellulose, modified cellulose, hemicellulose, starch or modified starch; phenolic macromolecules such as lignin; polyglucosamines such as chitosan, polyvinyl esters such as polyvinyl alcohols and polyvinyl acetate; phosphazenes, and polyesters such as polylactide or polyhydroxyalkanoate. This listing of specific components in the individual material classes is only exemplary and should not be understood as limiting. The skilled person is familiar with suitable natural wall formers. Furthermore, the skilled person is familiar with the various methods of wall formation, such as coacervation or interfacial polymerization.

The biopolymers can be selected according to the respective application in order to form a stable multi-layer shell with the material of the stability layer. Furthermore, the biopolymers can be selected to achieve compatibility with the chemical conditions of the application area. The biopolymers can be combined in various ways to influence the biodegradability or, for example, the stability and chemical resistance of the microcapsule.

According to one embodiment of the first aspect, the shell of the microcapsules exhibits a biodegradability of 50% according to OECD 301 F. In another embodiment, the shell of the microcapsule exhibits a biodegradability of at least 60% (OECD 301 F). In another embodiment, the biodegradability is at least 70% (OECD 301 F). The biodegradability is measured over a period of 60 days. In the extended biodegradation process (‘enhanced ready biodegradation’), the biodegradability is measured over a period of 60 days (see Opinion on an Annex XV dossier proposing restrictions on intentionally-added microplastics of Jun. 11, 2020, ECHA/RAC/RES-O-0000006790-71-01/F). The microcapsules are preferably freed from residues by washing before determining the biodegradability. Particularly preferred are copies of the microcapsules for this test made with an inert, non-biodegradable core material like perfluorooctane (PFO) instead of perfume oil. In one embodiment, the capsule dispersion is washed after production by threefold centrifugation and redispersion in distilled water. For this, the sample is centrifuged (e.g., for 10 min at 12,000 RPM). After removing the clear supernatant, it is filled with water and the sediment is redispersed by shaking. Various reference samples can be used in the measurement of biodegradability, such as the rapidly degradable ethylene glycol or natural-based walnut shell flour with the typical step-like degradation of a complex mixture of substances. The microcapsule according to the invention shows similar, preferably better, biodegradability over a period of 28 or 60 days than the walnut shell flour.

Residues in the microcapsule dispersions are substances used in the manufacture of the microcapsules and interact non-covalently with the shell, such as deposition aids, preservatives, emulsifiers/protective colloids, and excess ingredients. These residues have a proven impact on the biodegradability of microcapsule dispersions. Therefore, washing before determining the biodegradability is necessary.

To gain an understanding of the proportion of covalently and non-covalently bound components in the microcapsule dispersion, the capsules were analyzed using the quantification method based on Py-GC-MS for polymer-encapsulated fragrances as described in Gasparini et al. 2020 (Gasparini G, Semaoui S, Augugliaro J, Boschung A, Berthier D, Seyfried M, Begnaud F. Quantification of Residual Perfume by Py-GC-MS in Fragrance Encapsulate Polymeric Materials Intended for Biodegradation Tests. Molecules. 2020; 25(3):718). This method involves a multi-step cleaning protocol for polymers from complex samples like microcapsule dispersions and allows the quantification of volatile residual components, which are presumed not to be covalently bound into the 3D polymer network and are therefore not quantifiable with other standard methods (e.g., SPME-GC-MS or TGA). This procedure confirmed that individual layers of the microcapsule according to the invention, particularly the barrier and stability layers, are inseparably connected and can be considered as a monopolymer. It is assumed that the addition of the emulsion stabilizer not only improves the structural integration of the stability layer by the barrier layer but also increases the structural (covalent) connection of all wall-forming components.

A high value of biodegradability according to the invention is achieved not only by the wall formers used but also by the inventive structure of the shell. Because the use of a certain percentage of biopolymers does not automatically lead to a corresponding value of biodegradability. This depends on how the biopolymers are present in the shell

According to a preferred embodiment, the stability layer contains gelatin as a biopolymer. According to another preferred embodiment, the stability layer contains alginate as a biopolymer. According to another preferred embodiment, the stability layer contains gelatin and alginate as biopolymers. As shown in the exemplary embodiment, both gelatin and alginate are suitable for the production of inventive microcapsules with high biodegradability and high stability. In particular, it was shown that in a stability layer containing gelatin and alginate, treating the surface of the barrier layer with an emulsion stabilizer, especially a copolymer containing AMPS, leads to a significant increase in the layer thickness of the stability layer (see Examples 1-4). Other suitable combinations of natural components in the stability layer are gelatin and gum arabic.

The stability layer contains one or more curing agents. Curing agents according to the invention are aldehydes such as glutaraldehyde, formaldehyde, and glyoxal, as well as tannins, enzymes like transglutaminase, and organic anhydrides like maleic anhydride, epoxy compounds, polyvalent metal cations, amines, polyphenols, maleimides, sulfides, phenoloxides, hydrazides, isocyanates, isothiocyanates, N-hydroxysulfosuccinimide derivatives, carbodiimide derivatives, and polyols. Glutaraldehyde is preferred as a curing agent due to its very good crosslinking properties. Glyoxal is also preferred as a curing agent due to its good crosslinking properties and, compared to glutaraldehyde, lower toxicological classification. The use of curing agents achieves a higher impermeability of the stability layer. However, curing agents lead to reduced biodegradability of the natural polymers.

According to one embodiment, the barrier layers do not contain isocyanates. Some isocyanates, such as methylenediphenyl isocyanate (MDI), hexamethylene diisocyanate (HDI), and toluene-2,4-diisocyanate (TDI), exhibit a certain level of toxicity and are critically evaluated from an occupational safety perspective. Furthermore, side reactions with components of the core material can also occur with isocyanates.

According to one embodiment, the barrier layers according to the invention do not contain silane monomers, silane oligomers, or silicates. These components can be detrimental in certain combinations for the formation of the inventive capsule. For example, it is known to the skilled person that silicates like TEOS and TMOS (tetraethyl orthosilicate or tetramethyl orthosilicate) can react with components of an oil phase, such as fragrances, when added, thus negatively affecting the properties of the oil phase, which is the core material (e.g., fragrance oil).

Furthermore, TEOS and TMOS are considered critical from an occupational safety standpoint due to their flammability and toxicity and are preferably not used according to the invention.

According to one embodiment, the barrier layers do not contain silicone-melamine-polyurethane copolymer. Even with a silicone-melamine-polyurethane copolymer, side reactions can occur with the core material, i.e., the oil phase, especially with the fragrances contained therein. Additionally, a silicone-melamine-polyurethane copolymer is also considered critical from an occupational safety perspective.

Due to the presence of the barrier layer as a diffusion barrier, the amount of curing agent in the stability layer can be kept low, which in turn contributes to the easy biodegradability of the layer. According to one embodiment, the proportion of the curing agent in the stability layer is below 25 wt. %. Unless explicitly defined otherwise, the proportions of the components of the layers refer to the total weight of the layer, i.e., the total dry weight of the components used for production, excluding the components used in the production that are not or only marginally incorporated into the layer, such as surfactants and protective colloids. Above this value, the biodegradability according to OECD 301 F as per the invention cannot be guaranteed. The proportion of the curing agent in the stability layer can be, for example, 1.0 wt. %, 2.0 wt. %, 3.0 wt. %, 4.0 wt. %, 5.0 wt. %, 6 wt. %, 7 wt. %, 8 wt. %, 9 wt. %, 10 wt. %, 11 wt. %, 12 wt. %, 13 wt. %, 14 wt. %, 15 wt. %, 16 wt. %, 17 wt. %, 18 wt. %, 19 wt. %, 20 wt. %, 21 wt. %, 22 wt. %, 23 wt. %, or 24 wt. %. Preferably, the proportion of the curing agent in the stability layer is in the range of 1 to 15 wt. %. This proportion leads to effective crosslinking of the gelatin and results in a quantitative reaction that produces as little residual monomer as possible. The range of 4 to 12 wt. % is particularly preferred; it provides the required degree of crosslinking and a stable coating of the barrier layer to buffer the otherwise sensitive barrier layer and has little residual aldehyde, which is decomposed in a subsequent alkaline adjustment of the slurry via an aldol reaction.

According to one embodiment, the stability layer according to the invention contains gelatin and glutaraldehyde. According to another embodiment, the stability layer according to the invention contains gelatin, alginate, and glutaraldehyde. In an additional embodiment, the stability layer according to the invention contains gelatin and glyoxal. According to another embodiment, the stability layer according to the invention contains gelatin, alginate, and glyoxal. The exact chemical composition of the stability layer is not crucial. However, the effect according to the invention is preferably achieved with polar biopolymers.

The use of the emulsion stabilizer according to the invention on the surface of the barrier layer significantly increases the average thickness of the stability layer. The average thickness of the stability layer is at least 1 μm. The average thickness of the stability layer can be 1 μm, 1.2 μm, 1.4 μm, 1.6 μm, 1.8 μm, 2 μm, 2.2 μm, 2.4 μm, 2.6 μm, 2.8 μm, 3 μm, 3.5 μm, 4 μm, 4.5 μm, 5 μm, 5.5 μm, 6 μm, 6.5 μm, 7 μm, 7.5 μm, 8 μm, 8.5 μm, 9 μm, 9.5 μm, or 10 μm. The stability layer often has an elliptical shape in cross-section, so the thickness of the stability layer varies over the microcapsule surface. Therefore, an average thickness of the microcapsules is calculated. Additionally, the deposition varies from microcapsule to microcapsule. This is taken into account by determining the average thicknesses of several microcapsules and calculating the average from these. Thus, the average thickness mentioned here is actually an average of average thicknesses. The determination of the layer thickness of the stability layer can be done in two ways according to the invention. First, the light microscopic approach is mentioned here, i.e., the direct, optical measurement of the observed layer thickness using a microscope and corresponding software. A large number of microcapsules from a dispersion are measured, and due to the variance within the capsules, at least the diameter of each individual microcapsule is determined.

A second option is the measurement of the particle size distribution using laser diffraction. Here, the modal value of a particle size distribution without the layer to be measured can be compared to the modal value of a particle size distribution with the layer to be measured. The enlargement of this modal value indicates the increase in the hydrodynamic diameter of the main fraction of the measured microcapsules. Ultimately, the difference between the two measured modal values yields twice the layer thickness of the layer.

According to a preferred embodiment, the average thickness of the stability layer is at least 2 μm. By choosing a suitable combination of emulsion stabilizer and wall former of the stability layer, stability layers with an average thickness of 6 μm or more can be formed. In a particularly preferred embodiment, the average thickness of the stability layer is at least 3 μm.

In contrast to other biodegradable microcapsules, the microcapsules according to the invention exhibit high impermeability. According to one embodiment, the microcapsules have an impermeability that ensures the release of no more than 50 wt. % of the used core material after storage over a period of 4 weeks at a temperature of 0 to 40° C. As a result, the capsules are suitable for use in high-demand areas.

In various embodiments, the microcapsules according to the invention have an impermeability that ensures the release of no more than 80 wt. % of the used core material after storage over a period of 12 weeks at a temperature of 0 to 40° C., preferably no more than 75 wt. %, and more preferably no more than 70 wt. %. Therefore, in various embodiments, the microcapsules according to the invention still contain at least 20 wt. %, preferably at least 25 wt. %, and particularly at least 30 wt. % of the used core material after storage over a period of 12 weeks at a temperature of 0 to 40° C.

In further embodiments, the microcapsules according to the invention still contain at least 50 wt. % of the used core material after storage over a period of 4 weeks at a temperature of 0 to 40° C.

In addition to the shell material, the impermeability also depends on the type of core material. The impermeability of the microcapsules according to the invention was determined for the fragrance oil Weiroclean by Kitzing, as this fragrance oil is representative in its chemical properties for microencapsulated fragrance oils. Weiroclean has the following components (with the proportion relative to the total weight):

| | |
|---|---|
| 1-(1,2,3,4,5,6,7,8-Octahydro-2,3,8,8-tetramethyl-2- | 25-50% |
| naphthalenyl)ethanone | |
| Benzoic Acid, 2-hydroxy-, 2-hexyl ester | 10-25% |
| Phenylmethyl benzoate | 5-10% |
| 3-Methyl-4-(2,6,6-trimethyl-2-cyclohexenyl)-3-buten-2-one | 1-5% |
| 3,7-Dimethyl-6-octen-1-ol | 1-5% |
| 3-Methyl-5-phenylpentanol | 1-5% |
| 2,6-Dimethyloct-7-en-2-ol | 1-5% |
| 4-(2,6,6-Trimethylcyclohex-1-eneyl)-but-3-ene-2-one | 1-5% |
| 3a,4,5,6,7,7a-Hexahydro-4,7-methano-1H-inden-6-yl | 1-5% |
| Propanoate | |
| 2-tert-Butylcyclohexyl acetate | 1-5% |
| 2-Heptylcyclopentanone | 1-5% |
| Pentadecan-15-olide | 1-5% |
| 2H-1-Benzopyran-2-one | 0.1-1% |
| 2,6-Di-tert-butyl-p-cresol | 0.1-1% |
| 4-Methyl-3-decen-5-ol | 0.1-1% |
| 2,4-Dimethyl-3-cyclohexen-1-carboxaldehyde | 0.1-1% |
| [(2E)-3,7-dimethylocta-2,6-dienyl] acetate | 0.1-1% |
| Allyl hexanoate | 0.1-1% |
| 2-Methylundecanal | 0.1-1% |
| 10-Undecenal | 0.1-1% |
| cis-3,7-Dimethyl-2,6-octadienyl ethanoate | 0.1-1% |
| 3,7,11-Trimethyldodeca-1,6,10-trien-3-ol | 0.1-1% |
| Undecan-2-one | 0.1-1% |

A wide variety of materials can be used as the core material, including fragrances, flavors, phase change materials, cosmetic active ingredients, pharmaceutically active substances, catalysts, initiator systems, adhesive components, and hydrophobic reactive components. The core material in the microcapsules according to a preferred embodiment is hydrophobic. The core material can be solid or liquid, particularly liquid. Preferably, it is a liquid hydrophobic core material. In a preferred embodiment, the core material is a fragrance. Particularly preferred are fragrance oils optimized for microencapsulation in the laundry and cleaning agent sector, such as the fragrance formulation Weiroclean (Kurt Kitzing GmbH).

The impermeability of the capsule wall can be influenced by the choice of shell components. According to one embodiment, the microcapsules have an impermeability that ensures the release of no more than 45 wt. %, no more than 40 wt. %, no more than 35 wt. %, no more than 30 wt. %, no more than 25 wt. %, or no more than 20 wt. % of the used core material after storage over a period of 4 weeks at a temperature of 0 to 40° C. In various embodiments, the microcapsules still contain at least 55 wt. %, preferably at least 60 wt. %, more preferably at least 65 wt. %, even more preferably at least 70 wt. %, still more preferably at least 75 wt. %, and most preferably at least 80 wt. % of the used core material after storage over a period of 4 weeks at a temperature of 0 to 40° C.

The microcapsules are stored in a model formulation corresponding to the target application. Furthermore, the microcapsules are also stable in storage in the product in which they are used, such as in detergents, fabric softeners, cosmetics, adhesive systems, paints and dispersions, or in layered materials, such as coated papers. The standard formulations of these products are known to the skilled person. Typically, the pH value in the environment of the microcapsules during storage is in the range of 2 to 10.

The microcapsule shells according to the invention have at least two layers, meaning they can be, for example, double-layered, triple-layered, quadruple-layered, or quintuple-layered. Preferably, the microcapsules are double- or triple-layered.

According to one embodiment, the microcapsule has a third layer arranged on the outside of the stability layer. This third layer can be used to adapt the surface properties of the microcapsule for a specific application. This includes improving the adhesion of the microcapsules to various surfaces and reducing agglomeration. Additionally, the third layer binds residual aldehyde amounts, thereby reducing the content of free aldehydes in the capsule dispersion. Furthermore, it can provide additional (mechanical) stability or further increase the impermeability. Depending on the application, the third layer may contain a component selected from amines, organic salts, inorganic salts, alcohols, ethers, polyphosphazenes, and noble metals.

Noble metals increase the impermeability of the capsules and can impart additional catalytic properties to the microcapsule surface or enhance the antibacterial effect, for example, of a silver layer. Organic salts, particularly ammonium salts, lead to cationization of the microcapsule surface, resulting in better adhesion to, for example, textiles. Alcohols, when integrated via free hydroxyl groups, also lead to the formation of hydrogen bonds, which allow better adhesion to substrates. An additional polyphosphazene layer or coating with inorganic salts, such as silicates, leads to further increase in impermeability without affecting biodegradability. According to a preferred embodiment, the third layer contains activated melamine. The melamine captures any possible free aldehyde portions of the stability and/or barrier layer, increases the impermeability and stability of the capsule, and can also influence the surface properties of the microcapsules and thus the adhesion and agglomeration behavior.

Due to the low wall thicknesses, the proportion of the barrier layer in the shell relative to the total weight of the shell is at most 30 wt. %. The proportion of the barrier layer in the shell relative to the total weight of the shell can be, for example, 30 wt. %, 28 wt. %, 25 wt. %, 23 wt. %, 20 wt. %, 18 wt. %, 15 wt. %, 13 wt. %, 10 wt. %, 8 wt. %, or 5 wt. %. For high biodegradability, the proportion is at most 25 wt. % relative to the total weight of the shell. More preferably, the proportion of the barrier layer is at most 20 wt. %. For high biodegradability, the proportion is a maximum of 25 wt. % relative to the total weight of the shell. Particularly preferred is the proportion of the barrier layer being a maximum of 20 wt. %. The proportion of the stability layer in the shell, relative to the total weight of the shell, is at least 40 wt. %. The proportion of the stability layer in the shell, relative to the total weight of the shell, can be, for example, 40 wt. %, 43 wt. %, 45 wt. %, 48 wt. %, 50 wt. %, 53 wt. %, 55 wt. %, 58 wt. %, 60 wt. %, 63 wt. %, 65 wt. %, 68 wt. %, 70 wt. %, 75 wt. %, 80 wt. %, 85 wt. %, or 90 wt. %. For high biodegradability, the proportion of the stability layer is at least 50 wt. %, particularly preferred at least 60 wt. %. The proportion of the third layer in the shell, relative to the total weight of the shell, is a maximum of 35 wt. %. The proportion of the third layer in the shell, relative to the total weight of the shell, can be, for example, 35 wt. %, 33 wt. %, 30 wt. %, 28 wt. %, 25 wt. %, 23 wt. %, 20 wt. %, 18 wt. %, 15 wt. %, 13 wt. %, 10 wt. %, 8 wt. %, or 5 wt. %. For high biodegradability, the proportion of the third layer is preferably a maximum of 30 wt. %, particularly preferred a maximum of 25 wt. %.

The size of the microcapsules according to the invention is within the typical range for microcapsules. The diameter can range from 100 nm to 1 mm. The diameter depends on the exact capsule composition and the manufacturing process. The peak maximum of the particle size distribution is regularly used as a measure for the size of the capsules. Preferably, the peak maximum of the particle size distribution is in the range of 1 μm to 500 μm. The peak maximum of the particle size distribution can be, for example, at 1 μm, 2 μm, 3 μm, 4 μm, 5 μm, 10 μm, 15 μm, 20 μm, 30 μm, 40 μm, 50 μm, 60 μm, 70 μm, 80 μm, 90 μm, 100 μm, 120 μm, 140 μm, 160 μm, 180 μm, 200 μm, 250 μm, 300 μm, 350 μm, 400 μm, 450 μm, or 500 μm. In a particularly preferred embodiment, the microcapsules have a peak maximum of the particle size distribution of 10 μm to 100 μm. Particularly, the peak maximum of the particle size distribution is in the range of 10 μm to 50 μm.

The use of the emulsion stabilizer for coating the barrier layer represents a new application, distinct from the usual use of the emulsion stabilizer, namely stabilizing the core material droplets.

Therefore, according to a second aspect, the invention relates to the use of an emulsion stabilizer to increase the depositable amount of a stability layer on the surface of a barrier layer, where the barrier layer and the stability layer form the capsule wall of a microcapsule. The emulsion stabilizer and all other components of the microcapsules can be as defined in the first aspect.

### Use of Microcapsules and Product

Due to the robustness or impermeability of these biodegradable capsules, they can be used in a variety of products. Consequently, according to a third aspect, the invention relates to a product containing microcapsules according to the first aspect. In particular, the product is not from the field of laundry and cleaning agents and cosmetic products. The product can be an adhesive system; a pharmaceutical product; a coating material, particularly coated paper; a thermal storage coating, a self-healing coating, or a corrosion coating; or a coating containing microcapsules for functional packaging materials.

Furthermore, the invention, in a fourth aspect, relates to the use of microcapsules according to the first aspect for the manufacture of a product according to the third aspect.

In other words, the microcapsules can be used in the manufacturing of such a product. Consequently, the invention also relates to the use of the microcapsules according to the first aspect for the manufacturing of the product, where the product is selected from the group consisting of an adhesive system; a pharmaceutical product; a coating material, particularly coated paper; a thermal storage coating, a self-healing coating, or a corrosion coating; and such microcapsules containing for functional packaging materials.

### Manufacturing Method

Methods for manufacturing of core/shell microcapsules are known to the skilled person. Typically, an oil-based core material, which is not or scarcely water-soluble, is emulsified or dispersed in an aqueous phase containing the wall formers. Depending on the viscosity of liquid core materials, various units from simple stirrers to high-performance dispersers are used to distribute the core material into fine oil droplets. In this process, the wall formers separate from the continuous water phase onto the surface of the oil droplets and can then be crosslinked.

This mechanism is used in the in-situ polymerization of amino and phenoplast microcapsules and in the coacervation of water-soluble hydrocolloids.

In contrast, oil-soluble acrylate monomers are used for wall formation in radical polymerization. Furthermore, processes are used in which water-soluble and oil-soluble starting materials are reacted at the phase boundary of the emulsion droplets to form the solid shell.

Examples include the reaction of isocyanates and amines or alcohols to form polyurethane or polyurethane walls (interfacial polymerization), as well as the hydrolysis of silicate precursors followed by condensation to form an inorganic capsule wall (sol-gel process).

In a fifth aspect, the invention relates to a method for manufacturing microcapsules, comprising a fragrance as the core material and a shell consisting of three layers. During the production, the barrier layer serving as a diffusion barrier is provided as a template. To build this barrier layer, very small amounts of the aforementioned types of wall formers are needed. Preferably, the sensitive templates, after droplet formation at high stirring speeds, are equipped with an electrically negative charge by suitable protective colloids (e.g., Poly-AMPS) so that neither Ostwald ripening nor coalescence can occur. After creating this stable emulsion, with now greatly reduced stirring speed, the wall former, such as a suitable precondensate based on aminoplast resin, can form a very thin shell (layer). The thickness of the shell can be further reduced by the addition of an aromatic alcohol, e.g., m-aminophenol. This is followed by the formation of a production-capable shell structure, which surprisingly displays good affinity to biopolymers such as gelatin or alginate and allows deposition on the templates without the expected problems such as gelling of the mixture, agglomeration formation, and incompatibility of the structure provider.

As shown in the examples, the use of the emulsion stabilizer according to the invention further increased the deposition of the biopolymers.

The method comprises at least the following steps:
a) a) Creating an oil-in-water emulsion by emulsifying a core material in an aqueous phase in the presence of the wall-forming component(s) of the inner barrier layer, with the addition of protective colloids;
b) Deposition and curing of the wall-forming component(s) of the barrier layer, wherein the wall-forming component(s) of the barrier layer preferably are an aldehydic component, an amine component, and an aromatic alcohol, particularly preferably formaldehyde, melamine, and resorcin;
c) Adding an emulsion stabilizer, wherein the emulsion stabilizer is preferably as defined according to the first aspect;
d) Adding the wall-forming component(s) of the stability layer, followed by deposition and curing, wherein the wall-forming components of the stability layer are at least one biopolymer, preferably a protein and/or a polysaccharide, particularly preferably gelatin and alginate, as well as a curing agent, preferably glutaraldehyde or glyoxal; and
e) Optionally adding the wall-forming component(s) of the outer, third shell layer, followed by deposition and curing, wherein the wall-forming component(s) of the outer, third shell layer preferably are an amine component, particularly melamine.

In various process steps, the addition of a thickening agent, such as Jaguar HP105 (Solvay), can be advantageous. The thickening agent is particularly used for adjusting the viscosity. An increase in viscosity, for example, up to a viscosity of 2500 mPas (measured with Brookfield, RT, S3), can stabilize the microcapsule dispersion and thus improve its stirrability and storage.

The addition of the emulsion stabilizer is preferably done slowly over at least two minutes. According to one embodiment, the microcapsule dispersion is stirred. For stirring, a blade stirrer can be used, for example. The stirring speed is preferably in the range of 150 to 250 rpm. Above 250 rpm, there is a risk of air being entrained into the microcapsule dispersion. Below 150 rpm, the mixing might not be sufficient.

The temperature is preferably in the range of 15° C. to 35° C. The temperature can be at 15° C., 18° C., 20° C., 23° C., 25° C., 28° C., 30° C., 33° C., or 35° C. Particularly preferred is a temperature of 25° C. After addition, the microcapsule dispersion is stirred until a homogeneous mixture is formed. In one embodiment, the microcapsule dispersion is stirred for at least 5 minutes after addition. In a preferred embodiment, the microcapsule dispersion is stirred for at least 10 minutes after addition.

Alternatively, steps a) and b) can be carried out as follows:
a) Creating an oil-in-water emulsion by emulsifying a core material in an aqueous phase in the presence of the wall-forming component(s) of the inner barrier layer, optionally with the addition of protective colloids;
b) Deposition and curing of the wall-forming component(s) of the inner barrier layer, wherein the wall-forming component(s) of the inner barrier layer particularly are an aldehydic component, an amine component, and an aromatic alcohol.

This process can be carried out either sequentially or as a so-called one-pot process. In the sequential process, only steps a) and b) are carried out in a first procedure to obtain microcapsules with only the inner barrier layer as a shell (intermediate microcapsules). Then, a portion or the total amount of these intermediate microcapsules is transferred to another reactor. In this reactor, the further reaction steps are carried out. In the one-pot process, all process steps are carried out in a batch reactor. The implementation without reactor change is particularly time-saving.

For this, the entire system should be adjusted to the one-pot process. The correct choice of solid contents, the correct temperature management, the coordinated addition of formulation components, and the sequential addition of wall formers is thus possible.

According to one embodiment of the method, the process comprises the preparation of a water phase by dissolving a protective colloid, particularly a polymer based on acrylamidosulfonate and a methylated pre-polymer in water. The pre-polymer is preferably generated by reacting an aldehyde with either melamine or urea. Optionally, methanol can be used.

Furthermore, in the method according to the invention, the water phase can be mixed by stirring and setting a first temperature, where the first temperature is in the range of 30° C. to 40° C. Subsequently, an aromatic alcohol, particularly phloroglucin, resorcin, or aminophenol can be added to and dissolved in the water phase.

Alternatively, in the method according to the invention, an oil phase can be produced by mixing a fragrance composition or a phase change material (PCM) with aromatic alcohols, particularly phloroglucin, resorcin, or aminophenol. Alternatively, reactive monomers or diisocyanate derivatives can be introduced into the fragrance composition. This is followed by setting the first temperature.

Another step can be the preparation of a two-phase mixture by adding the oil phase to the water phase and then increasing the rotation speed.

Subsequently, emulsification can be started by adding formic acid. A regular determination of the particle size is advisable here. Once the desired particle size is reached, the two-phase mixture can be further stirred, and a second temperature can be set for curing the capsule walls. The second temperature can be in the range of 55° C. to 65° C.

This is followed by the addition of a melamine dispersion to the microcapsule dispersion and setting a third temperature, wherein the third temperature is preferably in the range of 75° C. to 85° C.

Another suitable step is the addition of an aqueous urea solution to the microcapsule dispersion. Subsequently, the emulsion stabilizer is added to the microcapsule dispersion before it is added to a solution of gelatin and alginate for the production of the stabilization layer.

In this case, the process would be followed by a cooling to a temperature between 45° C. and 55° C., and adjusting the pH of the microcapsule dispersion to a value in the range of 3.5 to 4.1, particularly 3.7.

Then, the microcapsule dispersion can be cooled to a fourth temperature, where the fourth temperature is in the range of 20° C. to 30° C. Subsequently, it can be cooled to a fifth temperature, where the fifth temperature is in a range of 4° C. to 17° C., particularly at 8° C.

Subsequently, the pH of the microcapsule dispersion would be adjusted to a value in the range of 4.3 to 5.1 and glutaraldehyde or glyoxal would be added. The reaction conditions, especially temperature and pH value, can be chosen differently depending on the crosslinker. The respective suitable conditions can be derived by the skilled person, for example, from the reactivity of the crosslinker. The amount of glutaraldehyde or glyoxal added influences the crosslinking density of the stability layer and thus, for example, the impermeability and degradability of the microcapsule shell. Accordingly, the expert can vary the amount to tailor the property profile of the microcapsule. To create the additional third layer, a melamine suspension consisting of melamine, formic acid, and water can be prepared. This is followed by the addition of the melamine suspension to the microcapsule dispersion. Finally, the pH of the microcapsule dispersion would be adjusted to a value in the range of 9 to 12, particularly 10 to 11.

Furthermore, for curing in step e), the microcapsule dispersion can be heated to a temperature in the range of 20° C. to 80° C. As shown in Example 8, this temperature has an influence on the color stability of the microcapsules. The temperature can be 20° C., 25° C., 30° C., 35° C., 40° C., 45° C., 50° C., 55° C., 60° C., 65° C., 70° C., 75° C., or 80° C. Below a temperature of 20° C., no influence on color stability is expected. A temperature above 80° C. could negatively affect the properties of the microcapsules. According to one embodiment, the temperature is in the range of 30° C. to 60° C. According to a preferred embodiment, the temperature is in the range of 35° C. to 50° C.

According to one embodiment, the microcapsule dispersion is maintained at the heating temperature for a period of at least 5 minutes. The duration can be, for example, 10 minutes, 15 minutes, 20 minutes, 25 minutes, 30 minutes, 35 minutes, 40 minutes, 45 minutes, 50 minutes, 55 minutes, 60 minutes, 70 minutes, 80 minutes, 90 minutes, 100 minutes, 110 minutes, or 120 minutes. According to one embodiment, the microcapsule dispersion is held at the heating temperature for a period of at least 30 minutes. According to another embodiment, the microcapsule dispersion is maintained at the heating temperature for a period of at least 60 minutes.

### Microcapsule Dispersion and Coloration

Microcapsules are typically present in the form of microcapsule dispersions. Therefore, the present invention also relates to microcapsule dispersions containing microcapsules according to the first aspect. Despite the use of aromatic alcohol in the barrier layer of the microcapsule shell, the microcapsule dispersions according to the invention show only slight coloring.

To qualify the discoloration, the color coordinate in the L*a*b* color space was determined for the microcapsules according to the invention. The L*a*b* color model is standardized in EN ISO 11664-4 “Colorimetry—Part 4: CIE 1976 L*a*b* Colour space”. The L*a*b* color space (also known as CIELAB, CIEL*a*b*, Lab colors) describes all perceptible colors. It uses a three-dimensional color space, where the brightness value L* is perpendicular to the color plane (a*,b*). The a-coordinate indicates the color type and color intensity between green and red, and the b-coordinate indicates the color type and color intensity between blue and yellow. The larger the positive a- and b-values and the smaller the negative a- and b-values, the more intense the color tone. If a=0 and b=0, it is an achromatic color tone on the brightness axis. The properties of the L*a*b* color model include device independence and perception relevance, meaning colors are defined regardless of their method of production or reproduction technique, as they are perceived by a standard observer under standard lighting conditions.

As shown in the examples, the microcapsule dispersions according to the invention in the L*a*b* color space have a color coordinate with an L* value of at least 50. The L* value can be, for example, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80. According to a preferred embodiment, the microcapsule dispersions according to the invention in the L*a*b* color space have a color coordinate with an L* value of at least 50. Particularly preferred is a color coordinate of at least 60.

Furthermore, the microcapsule dispersions produced by the manufacturing method according to the invention are particularly color—stable. As shown in the examples, the color coordinate of the microcapsule dispersion in the L*a*b* color space after storage has an L* value of at least 50. The L* value after storage can be, for example, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80. According to a preferred embodiment, the microcapsule dispersions according to the invention have a color coordinate in the L*a*b* color space with an L* value of at least 60 after storage. Particularly preferred is a color coordinate of at least 65.

The microcapsule dispersion may contain a thickening agent, such as Jaguar HP105 (Solvay). The thickening agent is particularly used for adjusting the viscosity. An increase in viscosity, for example, up to a viscosity of 2500 mPas (measured with Brookfield, RT, S3), can stabilize the microcapsule dispersion and thus improve its stirrability and storage.

According to one embodiment, the storage time is at least four weeks, preferably at least six weeks, and particularly at least eight weeks.

### EXAMPLES

### Example 1-Production of Reference Microcapsules not According to the Invention with Melamine-Formaldehyde Formulation MK2

### 1.1 Materials

The materials used for the production of the reference microcapsules-melamine-formaldehyde—are shown in Table 1.

TABLE 1
List of substances used for the production of MK2

| | | Concen- | MK2 |
|---|---|---|---|
| | | tration/ | Weight/ |
| Trade Name | Substance Name | wt. % | g |
| — | Deionized water | 100 | 187.5 |
| Dimension ™ SD, | 1,3,5-Triazine-2,4,6- | 67 | 42.5* |
| Solenis | triamine, polymer | | |
| | with formaldehyde, | | |
| | methylated (Content | | |
| | (W/W): >=60%-<=80%), | | |
| | in water | | |
| Dimension ™ SD, | Polymer based on | 20 | 35.0* |
| Solenis | acrylamidosulfonate | | |
| Weiroclean, Fa. Kurt | Core material (e.g., | 100 | 192.5* |
| Kitzing GmbH | fragrance oil, PCM, | | |
| | etc.) | | |
| — | Formic acid | 10 | 8.8 |
| Melafine ®, OCl | Melamine | 27 | 48.8 |
| Nitrogen B.V. | suspension | | |
| — | Urea solution | 28.6 | 70.0 |

1) Concentration refers to the acidified suspension..
*Quantities of the components refer to the commercial product and are used as supplied.

### 1.2 Manufacturing Process (Based on BASF Patent EP 1 246693 B1

Dimension SD was stirred into deionized water, after which Dimension PA140 was added and stirred until a clear solution was formed. The solution was heated to 30-35° C. in a water bath. While stirring with a dissolver disc, the perfume oil was added at 1100 rpm. The pH value of the oil-in-water emulsion was adjusted to 3.3-3.8 with a 10% formic acid solution. Then, the emulsion was stirred for another 30 minutes at 1100 rpm until a droplet size of 20-30 μm was reached, or the stirring was extended until the desired particle size of 20-30 μm (Peak-Max) was achieved. The particle size was determined using a Beckmann-Coulter device (laser diffraction, Fraunhofer method). The rotation speed was reduced depending on the viscosity to ensure good mixing. With this rotation speed, the mixture was stirred for another 30 minutes at 30 to 40° C. Subsequently, the emulsion was heated to 60° C. and stirred further. The melamine suspension was adjusted to a pH value of 4.5 with formic acid (10%) and added to the reaction mixture. The mixture was maintained at 60° C. for 60 minutes and then heated to 80° C. After stirring for 60 minutes at 80° C., the urea solution was added.

After cooling to room temperature, the microcapsule dispersion was filtered through a 200-μm filter sieve.

### 1.3 Result

The obtained MF reference microcapsule MK2 was examined microscopically. A typical image of MK2 is shown in FIG. 1F. To evaluate the obtained microcapsules, the pH value, solid content, viscosity, particle size, core material content in the slurry, and the L* value of the color location were determined. The result is shown in Table 2.

TABLE 2
Analysis results of the reference microcapsule
not according to the invention MK2

| | Measurement method | MK 2 |
|---|---|---|
| pH | pH electrode | 5.5-6.5 |
| Solid content/% | Microwave | 37-41 |
| Viscosity/mPas | Brookfield viscometer | <1000 |
| Particle size Peak max./μm | Laser diffraction | 20-30 |
| Core material content in | Calculation from recipe | 30-35 |
| slurry/% | | |
| L*-value/— | Spectrophotometer | 65-80 |

### Example 2-Production of Microcapsule Dispersions According the Invention Slurry 2 and Slurry 5 and Reference Microcapsule not According to the Invention MK1

### 2.1 Materials

The materials used for the production of the microcapsules according to the invention—Slurry 2 and Slurry 5 are shown in Table 3.

TABLE 3
List of substances used for the production of Slurry 2 and 5

| | | | Slurry 2 | Slurry 5 |
|---|---|---|---|---|
| | | Concentration/ | Weight/ | Weight/ |
| Trade Name | Substance Name | wt. % | g | g |
| | Deionized water addition 1 | 100 | 35.2 | 39.6 |
| Dimension ™ SD, | 1,3,5-Triazine-2,4,6- | 67 | 2.1* | 1.7* |
| Solenis | triamine, polymer with | | | |
| | formaldehyde, methylated | | | |
| | (Content | | | |
| | (W/W): >=60%-<=80%), | | | |
| | in water | | | |
| Dimension ™ PA 140, | Polymer based on | 20 | 4.6* | 3.8* |
| Solenis | acrylamidosulfonate | | | |
| | addition 1 | | | |
| Weiroclean, Kurt | Core material (e.g., | 100 | 52.8* | 52.6* |
| Kitzing GmbH | fragrance oil, PCM, etc.) | | | |
| — | Resorcin solution | 3.2 | 12.7 | 11.9 |
| — | Fomic acid addition 1 | 10 | 0.7 | 0.4 |
| Melafine ®, OCl | Melamine-suspension | 27 | 2.6 | 2.0 |
| Nitrogen B.V. | Addition 1 ¹⁾ | | | |
| — | Urea solution | 41.9 | 2.6 | 2.2 |
| Dimension ™ PA 140, | Polymer based on | 20 | 9.5* | 9.6* |
| Solenis | acrylamidosulfonate | | | |
| | addition 2 | | | |
| — | Tap water | 100 | 136.2 | 135.6 |
| | Sodium sulfate | 100 | 0.6* | 0.6* |
| Scogin ® MV, DuPont | Sodium alginate | 100 | 2.0* | 2.0* |
| Nutrition Ireland | | | | |
| Speisegelatinepulver, | Pork skin gelatin | 100 | 8.4* | 8.4* |
| Ewald-Gelatine GmbH | | | | |
| — | Formic acd addition 2 ²⁾ | 20 | 2.5 | 2.5 |
| — | Sodium hydroxide | 20 | 2.4 | 2.3 |
| | Addition 1 ²⁾ | | | |
| Glutaraldehyde, 50% aq. | Glutaraldehyde solution | 50 | 2.6* | 2.6* |
| soln., Alfa Aesar | | | | |
| Melafine ®, OCl | Melamine suspension | 27 | 27.5 | 27.4 |
| Nitrogen B.V. | addition 2 ¹⁾ | | | |
| — | Sodium hydroxide | 20 | 4.7 | 4.7 |
| | addition 2 ²⁾ | | | |

¹⁾ Concentration refers to the acidified suspension.
²⁾ The quantities given for acids/bases are guide values. It is adjusted to the pH range specified in the experimental procedure.
*Quantities of the components refer to the commercial product and are used as supplied.

The materials used for the production of the reference microcapsules MK1 are shown in Table 4.

TABLE 4
List of substances used for the production of MK1

| | | MK1 | |
|---|---|---|---|
| | | Concentration/ | Weight/ |
| Trade Name | Substance Name | wt. % | g |
| | Deionized water addition 1 | 100 | 34.9 |
| Dimension ™ SD, Solenis | 1,3,5-Triazine-2,4,6-triamine, | 67 | 1.6* |
| | polymer with formaldehyde, | | |
| | methylated (Content | | |
| | (W/W): >=60%-<= 80%), | | |
| | in water | | |
| Dimension ™ PA 140, | Polymer based on | 20 | 3.4* |
| Fa. Solenis | acrylamidosulfonate | | |
| Weiroclean, Kurt Kitzing | Core material (e.g., fragrance | 100 | 38.8* |
| GmbH | oil, PCM, etc.) | | |
| — | Resorcin solution | 12.2 | 2.5 |
| — | Formic acid addition 1 | 20 | 0.5 |
| Melafine ®, OCl Nitrogen | Melamin suspension | 27 | 1.9 |
| B.V. | addition 1 ¹⁾ | | |
| — | Urea solution | 16.6 | 4.7 |
| — | Tap water | 100 | 100.19 |
| — | Sodium sulfate | 100 | 0.5* |
| Speisegelatinepulver, | Pork skin gelatin | 100 | 6.2* |
| Ewald-Gelatine GmbH | | | |
| Scogin ® MV, DuPont | Sodium alginate | 100 | 1.4* |
| Nutrition Ireland | | | |
| — | Formic acid addition 2 ²⁾ | 20 | 1.4 |
| — | Sodium hydroxide addition 1 ²⁾ | 20 | 0.8 |
| Glutaraldehyde, 50% aq. | Glutaraldehyde solution | 50 | 1.9* |
| soln., Alfa Aesar | | | |
| Melafine ®, OCl Nitrogen | Melamine suspension addition | 27 | 6.7 |
| B.V. | 2 ¹⁾ | | |
| — | Sodium hydroxide addition 2 ²⁾ | 20 | 2.2 |

¹⁾ Concentration refers to the acidified suspension.
²⁾ The quantities given for acids/bases are guide values. It is adjusted to the pH range specified in the experimental procedure.
*Quantities of the components refer to the commercial product and are used as supplied.

### 2.2 Manufacturing Process for the Microcapsule not According to the Invention MK1

To prepare reaction mixture 1, Dimension PA140 and Dimension SD were weighed with deionized water (addition 1) in a beaker and pre-mixed using a 4 cm dissolver disc. The beaker was fixed in a water bath and stirred at 500 RPM at 30° C. with the dissolver disc until a clear solution was formed. Once the Dimension SD/Dimension PA140 solution became clear and reached 30-40° C., the perfume oil was slowly added while adjusting the stirring speed (1100 RPM) to achieve the desired particle size. The pH of this mixture was then acidified by adding formic acid (addition 1). The mixture was emulsified for 20-30 minutes, or extended as needed until the desired particle size of 20-30 μm (Peak-Max) was reached. The particle size was determined using a Beckman-Coulter device (laser diffraction, Fraunhofer method). After reaching the desired particle size, the stirring speed was reduced to ensure gentle mixing.

The resorcin solution was then stirred in and pre-formed gently for 30-40 minutes. After the pre-formation time, the emulsion temperature was raised to 50° C. within 15 minutes. Upon reaching this temperature, the mixture was heated to 60° C. over 15 minutes and maintained at this temperature for another 30 minutes. Then, using 20% formic acid, the pH of melamine suspension addition 1 was adjusted to 4.5 and dosed into the reaction mixture over 90 minutes. The temperature was maintained for 30 minutes. After this period, the temperature was first raised to 70° C. within 15 minutes, then to 80° C. within another 15 minutes, and held for 120 minutes. Afterward, the aqueous urea solution was added, the heat source turned off, and Reaction Mixture 1 cooled to room temperature. In a separate beaker, sodium sulfate was dissolved in tap water while stirring with a wing stirrer at 40-50° C. Sodium alginate and pork skin gelatin were slowly sprinkled into the heated water. After all solids dissolved, reaction mixture 1 was stirred into the prepared gelatin/sodium alginate solution. Once a homogeneous mixture was reached, the pH was adjusted to 3.9 with formic acid addition 2, and then the heat source was removed. The mixture was then cooled to room temperature. After reaching room temperature, the reaction mixture was cooled with ice. Upon reaching 8° C., the ice bath was removed, and the pH was raised to 4.7 with Sodium hydroxide addition 1. Glutaraldehyde was then added, ensuring that the temperature did not exceed 16-20° C. until its addition.

Then, the melamine suspension addition 2, acidified to a pH of 4.5 with 20% formic acid, was dosed slowly. The reaction mixture was then heated to 60° C. and maintained at this temperature for 60 minutes. After this holding time, the heat source was removed, and the microcapsule suspension was gently stirred for 14 hours. After 14 hours, the microcapsule suspension was adjusted to a pH of 10.5 with sodium hydroxide addition 2.

### 2.2.1 Result

The resulting microcapsule MK1 was examined microscopically. Typical images are shown in FIG. 1E. To evaluate MK1, the pH, solid content, viscosity, particle size, core material content in the slurry, and the L* value of the color locus were determined. The result is shown in Table 5.

TABLE 5
Analysis results of the microcapsule
not according to the invention MK1

| | Measurement method | MK 1 |
|---|---|---|
| pH | pH electrode | 8-10 |
| Solid content/% | Microwave | 20-30 |
| Viscosity/mPas | Brookfield viscometer | <1000 |
| Particle size Peak max./μm | Laser diffraction | 20-30 |
| Core material content in | Calculation from recipe | 15-20 |
| slurry/% | | |
| L*-value/— | Spectrophotometer | 55-75 |

### 2.3 Manufacturing Process for the Microcapsule Dispersions According to the Invention Slurry 2 Und Slurry 5

To prepare reaction mixture 1, Dimension PA140 addition 1 and Dimension SD were weighed with deionized water addition 1 in a beaker and pre-mixed with a 4 cm dissolver disc. The beaker was fixed in a water bath, and stirred with the dissolver disc at 500 rpm at 30° C. until a clear solution was formed.

As soon as the Dimension SD/Dimension PA140 solution was clear and reached 30-40° C., the core material was slowly added while adjusting the speed (e.g., 1100 rpm) to achieve the desired particle size. Then, the pH value of this mixture was acidified to pH=3.3-3.5 by adding formic acid addition 1.

It was emulsified for 20-30 min or extended until the desired particle size of 20-30 μm (Peak-Max) was reached. The particle size was determined using a Beckmann-Coulter device (laser diffraction, Fraunhofer method). After reaching the particle size, the speed was reduced to ensure gentle mixing and the resorcinol solution was added.

Under gentle stirring, it was preformed for 30-40 min. After the preforming time elapsed, the emulsion temperature was raised to 50° C. within 15 min. Upon reaching this temperature, the mixture was heated to 60° C. over a period of 15 min and this temperature was maintained for another 30 min. Then, the pH value of the Melafin suspension addition 1 was adjusted to 4.5 with 20% formic acid and added to the reaction mixture over a period of 90 min.

After that, the temperature was maintained for 30 min. After the 30 min, the temperature was first raised to 70° C. within 15 min. Then, the temperature was raised to 80° C. within another 15 min and maintained for 90 min.

Then, the aqueous urea solution was added, the heat source was turned off, and reaction mixture 1 was cooled to room temperature. After reaction mixture 1 reached room temperature, Dimension PA140 addition 2 was added.

In a separate beaker, sodium sulfate was dissolved in tap water under stirring with a paddle stirrer at 40-50° C. Sodium alginate and pork skin gelatin were slowly sprinkled into the heated tap water. After all solids were dissolved, reaction mixture 1 was stirred into the prepared gelatin/sodium alginate solution. When a homogeneous mixture was achieved, the pH value was adjusted to 3.7 by slowly adding formic acid addition 2, then the heat source was removed, and the mixture was naturally cooled to room temperature.

After reaching room temperature, the reaction mixture was cooled with ice. Upon reaching a temperature of 8° C., the ice bath was removed, and the pH value was raised to 4.7 with sodium hydroxide addition 1. Then, 50% glutaraldehyde was added, ensuring that the temperature did not exceed 16-20° C. until the addition of 50% glutaraldehyde.

Subsequently, the melamine suspension addition 2, acidified to a pH of 4.5 with 20% formic acid, was added over a period of about 2 min. The microcapsule suspension was then gently stirred at room temperature for 14 hours. After the 14 hours, the microcapsule suspension was adjusted to a pH of 10.5 with sodium hydroxide addition 2 over a period of about 15 min.

### 2.3.1 Result

The obtained microcapsules according to the invention, Slurry 2 and Slurry 5, were examined under a light microscope. Typical images are shown in FIGS. 1A and 1C. To evaluate Slurry 2 and Slurry 5, the pH value, solid content, viscosity, particle size, content of core material in the slurry, and the L* value of the color coordinate were determined. The result is shown in Table 6.

TABLE 6
Analysis results for the microcapsule dispersion
according to the invention Slurry 2 and Slurry 5

| | | Slurry 2 |
|---|---|---|
| | Measurement method | und Slurry 5 |
| pH | pH electrode | 8-10 |
| Solid content/% | Microwave | 20-30 |
| Viscosity/mPas | Brookfield viscometer | <1000 |
| Particle size Peak max./μm | Laser diffraction | 20-30 |
| Core material content in | Calculation from recipe | 15-20 |
| slurry/% | | |
| L*-value/— | Spectrophotometer | 61-67 |

### Example 3-Production of Microcapsule Dispersions According the Invention Slurry 3 and Slurry 6 and Reference Microcapsule not According to the Invention MK4

### 3.1 Materials

The materials used for the production of the microcapsules according to the invention—Slurry 3 and Slurry 6 are shown in Table 7.

TABLE 7
List of substances used for the production of Slurry 3 and 6

| | | Concen- | Slurry 3 | Slurry 6 |
|---|---|---|---|---|
| | Substance | tration/ | weight/ | weight/ |
| Trade Mark | Name | wt. % | g | g |
| — | Deionized water | 100 | 35.2 | 39.6 |
| | addition 1 | | | |
| Dimension ™ | 1,3,5-Triazine- | 67 | 2.1* | 1.7* |
| SD, Solenis | 2,4,6-triamine, | | | |
| | polymer with | | | |
| | formaldehyde, | | | |
| | methylated, | | | |
| | aqueous | | | |
| Dimension ™ | Polymer based | 20 | 4.6* | 3.8* |
| PA 140, | on | | | |
| Solenis | acrylamidosulfonate | | | |
| | addition 1 | | | |
| Weiroclean, | Core material | 100 | 52.8* | 52.6* |
| Kurt Kitzing | (e.g., fragrance | | | |
| GmbH | oil, PCM, etc.) | | | |
| — | Resorcinol | 3.2 | 12.7 | 11.9 |
| | solution | | | |
| — | Formic acid | 10 | 0.7 | 0.4 |
| | addition 1 | | | |
| Melafine ®, | Melamine | 27 | 2.6 | 2.0 |
| OCl Nitrogen | suspension | | | |
| B.V. | (pH = 4.5) | | | |
| | addition 1 ¹⁾ | | | |
| — | Urea solution | 41.9 | 2.6 | 2.2 |
| Dimension ™ | Polymer Based | 20 | 9.5* | 9.6* |
| PA 140, | on | | | |
| Solenis | acrylamidosulfonate | | | |
| | addition 2 | | | |
| — | Tap water | 100 | 136.2 | 135.6 |
| — | Sodium sulfate | 100 | 0.6* | 0.6* |
| Scogin ® MV, | Sodium alginate | 100 | 2.0* | 2.0* |
| DuPont | | | | |
| Nutrition | | | | |
| Ireland | | | | |
| Speisegelatin | Pork skin gelatin | 100 | 8.4* | 8.4* |
| epulver, | | | | |
| Ewald- | | | | |
| Gelatine | | | | |
| GmbH | | | | |
| — | Formic acid | 20 | 2.5 | 2.5 |
| | addition 2 ²⁾ | | | |
| — | Sodium hydroxide | 20 | 2.4 | 2.3 |
| | addition 11 ²⁾ | | | |
| Glyoxal 40% | Glyoxal | 40 | 1.9* | 1.9* |
| solution in | | | | |
| water for | | | | |
| synthesis, | | | | |
| Merck KGaA | | | | |
| Melafine ®, | Melamine | 27 | 27.5 | 27.4 |
| OCl Nitrogen | suspension | | | |
| B.V. | (pH = 4.5) addition | | | |
| | 2 ¹⁾ | | | |
| — | Sodium hydroxide | 20 | 4.7 | 4.7 |
| | addition 2 ²⁾ | | | |

¹⁾ Concentration refers to the acidified suspension.
²⁾ The quantities given for acids/bases are guide values. It is adjusted to the pH range specified in the experimental procedure.
*Quantities of the components refer to the commercial product and are used as supplied

The materials used for the production of the reference microcapsules MK4 are shown in Table 8.

TABLE 8
List of substances used for the production of MK4

| | | Concen- | MK4 |
|---|---|---|---|
| | | tration/ | Weight/ |
| Trade Name | Substance Name | wt. % | g |
| — | Deionized water | 100 | 34.9 |
| | addition 1 | | |
| Dimension ™ SD, | 1,3,5-Triazine-2,4,6- | 67 | 1.6* |
| Solenis | triamine, polymer with | | |
| | formaldehyde, | | |
| | methylated, aqueous | | |
| Dimension ™ PA | Polymer based on | 20 | 3.4* |
| 140, Solenis | acrylamidosulfonate | | |
| | addition 1 | | |
| Weiroclean, Kurt | Core material (e.g., | 100 | 38.8* |
| Kitzing GmbH | fragrance oil, PCM, | | |
| | etc.) | | |
| — | Resorcinol solution | 12.2 | 2.5 |
| — | Formic acid addition 1 | 20 | 0.5 |
| Melafine ®, OCl | Melamine suspension | 27 | 1.9 |
| Nitrogen B.V. | (pH = 4.5) addition 1 ¹⁾ | | |
| — | Urea solution | 16.6 | 4.7 |
| — | Tap water | 100 | 100.2 |
| — | Sodium sulfate | 100 | 0.5* |
| Scogin ® MV, | Sodium alginate | 100 | 1.4* |
| DuPont Nutrition | | | |
| Ireland | | | |
| Speisegelatinepulver, | Pork skin gelantin | 100 | 6.2* |
| Ewald-Gelatine | | | |
| GmbH | | | |
| — | Formic acid addition | 20 | 1.4 |
| | 2 ²⁾ | | |
| — | Sodium hydroxide | 20 | 0.8 |
| | addition 1 ²⁾ | | |
| Glyoxal 40% | Glyoxal | 40 | 1.4* |
| solution in water | | | |
| for synthesis, | | | |
| Merck KGaA | | | |
| Melafine ®, OCl | Melamine suspension | 27 | 6.7 |
| Nitrogen B.V. | (pH = 4.5) addition 2 ¹⁾ | | |
| — | Sodium hydroxide | 20 | 2.2 |
| | addition 2 ²⁾ | | |

¹⁾ Concentration refers to the acidified suspension.
²⁾ The quantities given for acids/bases are guide values. It is adjusted to the pH range specified in the experimental procedure
*Quantities of the components refer to the commercial product and are used as supplied.

### 3.2. Manufacturing Process for the Microcapsule not According to the Invention MK4

To prepare reaction mixture 1, Dimension PA140 and Dimension SD were weighed with water addition 1 in a beaker and pre-mixed with a 4 cm dissolver disc. The beaker was fixed in a water bath, and stirred with the dissolver disc at 500 rpm at 30° C. until a clear solution was formed.

Once the Dimension SD/Dimension PA140 solution was clear and reached 30-40° C., the core material was slowly added and the speed was adjusted (e.g., 1100 rpm) to achieve the desired particle size. Then, the pH of this mixture was acidified by adding formic acid addition 1. It was emulsified for 20-30 min or extended until the desired particle size of 20-30 μm (Peak-Max) was reached. The particle size was determined using a Beckman-Coulter device (laser diffraction, Fraunhofer method). After reaching the particle size, the speed was reduced to ensure gentle mixing.

Then, the resorcinol solution was stirred in and preformed under gentle stirring for 30-40 min. After the preforming time, the emulsion temperature was increased to 50° C. within 15 min. Upon reaching this temperature, the mixture was heated to 60° C. over a period of 15 min and this temperature was maintained for another 30 min. Then, the pH of the melamine suspension addition 1 was adjusted to 4.5 with 20% formic acid and added to the reaction mixture over a period of 90 min. After that, the temperature was maintained for 30 min. After the 30 min, the temperature was first raised to 70° C. within 15 min. Then, the temperature was raised to 80° C. within another 15 min and maintained for 120 min. After that, the aqueous urea solution was added, the heat source was turned off, and reaction mixture 1 was cooled to room temperature. In a separate beaker, sodium sulfate was dissolved in tap water under stirring with a paddle stirrer at 40-50° C. Sodium alginate and pork skin gelatin were slowly sprinkled into the heated water. After all solids were dissolved, reaction mixture 1 was stirred into the prepared gelatin/sodium alginate solution. When a homogeneous mixture was achieved, the pH was adjusted to 3.9 with formic acid addition 2, then the heat source was removed. Afterwards, the mixture was cooled to room temperature. After reaching room temperature, the reaction mixture was cooled with ice. Upon reaching a temperature of 8° C., the ice bath was removed and the pH was raised to 4.7 with sodium hydroxide addition 1. Then, the glyoxal solution was added, ensuring that the temperature did not exceed 16-20° C. until the addition of the glyoxal solution. Subsequently, the melamine suspension addition 2, acidified to a pH of 4.5 with 20% formic acid, was slowly added. Then, the reaction mixture was heated to 60° C. and maintained at this temperature for 60 min. After this holding time, the heat source was removed and the microcapsule suspension was gently stirred for 14 hours. After the 14 hours, the pH of the microcapsule suspension was adjusted to 10.5 with sodium hydroxide addition 2.

### 3.2.1 Results

The obtained microcapsule MK4 was examined under a light microscope. Typical images are shown in FIG. 1G. For the evaluation of MK1, the pH value, solid content, viscosity, particle size, content of core material in the slurry, and the L* value of the color coordinate were determined. The result is shown in Table 9.

TABLE 9
Analysis results for the reference microcapsule
not according the invention MK4

| | Measurement method | MK4 |
|---|---|---|
| pH | pH electrode | 8-10 |
| Solid content/% | Microwave | 20-30 |
| Viscosity/mPas | Brookfield viscometer | <1000 |
| Particle size Peak max./μm | Laser diffraction | 20-30 |
| Core material content in | Calculation from recipe | 15-20 |
| slurry/% | | |
| L*-value/— | Spectrophotometer | 51-54 |

### 3.3. Manufacturing Process for the Microcapsule Dispersions According to the Invention Slurry 3 Und Slurry 6

To prepare reaction mixture 1, Dimension PA140 addition 1 and Dimension SD were weighed with deionized water addition 1 in a beaker and pre-mixed with a 4 cm dissolver disc. The beaker was fixed in a water bath, and stirred with the dissolver disc at 500 rpm at 30° C. until a clear solution was formed.

Once the Dimension SD/Dimension PA140 solution was clear and reached 30-40° C., the core material was slowly added and the speed was adjusted (e.g., 1100 rpm) to achieve the desired particle size. Then, the pH of this mixture was acidified to pH=3.3-3.5 by adding formic acid addition 1.

It was emulsified for 20-30 min or extended until the desired particle size of 20-30 μm (Peak-Max) was reached. The particle size was determined using a Beckman-Coulter device (laser diffraction, Fraunhofer method). After reaching the particle size, the speed was reduced to ensure gentle mixing and then the resorcinol solution was added.

Under gentle stirring, it was preformed for 30-40 min. After the preforming time, the emulsion temperature was raised to 50° C. within 15 min. Upon reaching this temperature, the mixture was heated to 60° C. over a period of 15 min and this temperature was maintained for another 30 min. Then, the pH of the melamine suspension addition 1 was adjusted to 4.5 with 20% formic acid and added to the reaction mixture over a period of 90 min.

After that, the temperature was maintained for 30 min. After the 30 min, the temperature was first raised to 70° C. within 15 min. Then, the temperature was raised to 80° C. within another 15 min and maintained for 90 min.

Then, the aqueous urea solution was added, the heat source was turned off, and reaction mixture 1 was cooled to room temperature. After reaction mixture 1 reached room temperature, Dimension PA140 addition 2 was added.

In a separate beaker, sodium sulfate was dissolved in tap water under stirring with a paddle stirrer at 40-50° C. Sodium alginate and pork skin gelatin were slowly sprinkled into the heated tap water. After all solids were dissolved, reaction mixture 1 was stirred into the prepared gelatin/sodium alginate solution. When a homogeneous mixture was achieved, the pH was adjusted to 3.7 with formic acid addition 2, then the heat source was removed and the mixture was naturally cooled to room temperature.

After reaching room temperature, the reaction mixture was cooled with ice to a temperature of 8° C. and the temperature was maintained at 8° C. The pH was raised to 4.7 with sodium hydroxide addition 1. Then, at a temperature of 8° C., 40% glyoxal was added, followed by the slow addition of the melamine suspension addition 2, acidified to a pH of 4.5 with 20% formic acid, over a period of about 2 min. The pH was adjusted to a value of pH=10.5 with sodium hydroxide addition 2 over a period of about 15 min. The ice bath was removed and the reaction mixture was heated to 40° C. and maintained at this temperature for 1 hour.

After this holding time, the microcapsule suspension was gently stirred at room temperature for 14 hours.

### 3.3.1 Result

The obtained microcapsule according to the invention, Slurry 3 and Slurry 6, were examined under a light microscope. Typical images are shown in FIGS. 1B and 1D. For the evaluation of Slurry 3 and Slurry 6, the pH value, solid content, viscosity, particle size, content of core material in the slurry, and the L* value of the color coordinate were determined. The result is shown in Table 10.

TABLE 10
Analysis results of the microcapsule dispersion
according to the invention Slurry 3 und Slurry 6

| | | Slurry 3 |
|---|---|---|
| | Measurement method | and Slurry 6 |
| pH | pH electrode | 6-10 |
| Solid content/% | Microwave | 20-30 |
| Viscosity/mPas | Brookfield viscometer | <1000 |
| Particle size Peak max./μm | Laser diffraction | 20-30 |
| Core material content in | Calculation from recipe | 15-20 |
| slurry/% | | |
| L*-value/— | Spectrophotometer | 55-59 |

### Example 4-Microscopic Determination of the Thickness of the Stability Layer

### 4.1 Preliminary Remarks

The determination of the layer thickness of the stability layer can in principle be done in two ways. First, there is the light microscopic approach, that is, the direct optical measurement of the observed layer thickness using a microscope and corresponding software.

A second possibility is the measurement of the particle size distribution using laser diffraction. Here, the modal value of a particle size distribution of the pure barrier template (see Example 1) can be compared to the modal value of a particle size distribution for a microcapsule according to the invention. The increase in this measurement value should reflect the increase in the hydrodynamic diameter (due to the application of the stability layer) of the main fraction of measured microcapsules. The formation of the difference between the two measured modal values ultimately gives the double layer thickness of the stability layer.

Both measurement methods provided consistent results in preliminary tests, which is why the following only describes the implementation and result of the light microscopic investigation.

### 4.2 Experimental Procedure

The determination of the layer thickness of the stability layer was carried out microscopically using an Olympus BX50 microscope. For measurement, the software OLYMPUS Stream Essentials 2.4.2 (Build 20105) was used.

Initially, a highly diluted sample of the capsule slurry according to the invention was prepared with tap water. A drop of this dispersion was applied to a slide and covered with a cover glass.

For determining the layer thickness, a magnification of 500× was selected on the microscope, and the corresponding microcapsules according to the invention in the applied sample dispersion were focused.

In the aforementioned software, the diameter of the visible barrier template was then captured using the “3-Point Circle” function. The layer thickness of the stability layer was ultimately measured at three characteristic points using the ruler function.

Due to the elliptical shape of the stability layer, several layer thickness measurements were taken for each focused microcapsule to represent the variance in layer thickness. The microcapsules according to the invention have a larger layer thickness at two opposing apex points and a smaller layer thickness at the other two opposing apex points. To reduce this measurement error in specifying a layer thickness for the stability layer, an average was calculated over 10 individual microcapsules.

### 4.3 Results

TABLE 11
Results of the layer thickness measurement of the
stability layer using Slurry 3 as an example

| No. of Considered | Diameter of Barrier | Layer Thickness/μm | | |
|---|---|---|---|---|
| Capsule/— | Template/μm | M1 | M2 | M3 |
| 1 | 30.48 | 3.02 | 7.86 | 4.02 |
| 2 | 26.12 | 4.74 | 3.37 | 4.36 |
| 3 | 25.78 | 4.76 | 4.25 | 3.21 |
| 4 | 25.81 | 5.74 | 4.53 | 2.23 |
| 5 | 24.28 | 8.12 | 2.38 | 7.17 |
| 6 | 16.08 | 2.28 | 3.72 | 1.73 |
| 7 | 30.86 | 3.75 | 3.21 | 5.56 |
| 8 | 20.37 | 2.03 | 3.18 | 4.01 |
| 9 | 36.83 | 3.23 | 4.68 | 3.21 |
| 10 | 20.74 | 3.86 | 2.04 | 3.54 |

| | |
|---|---|
| Mean | 3.99 |
| SD | 1.59 |

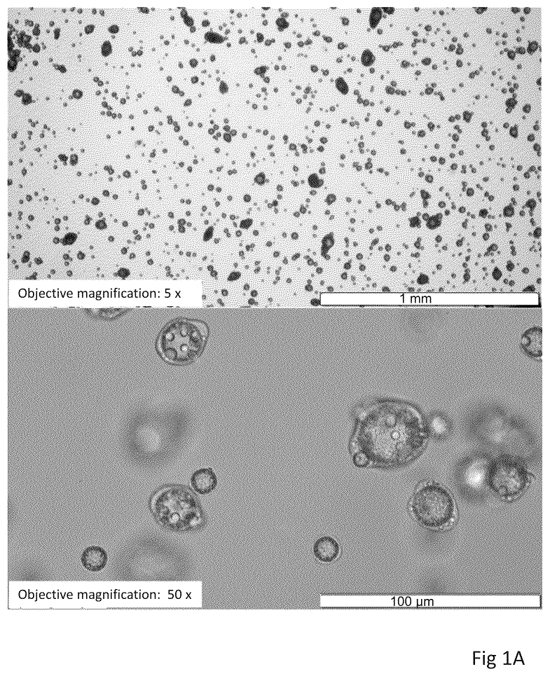

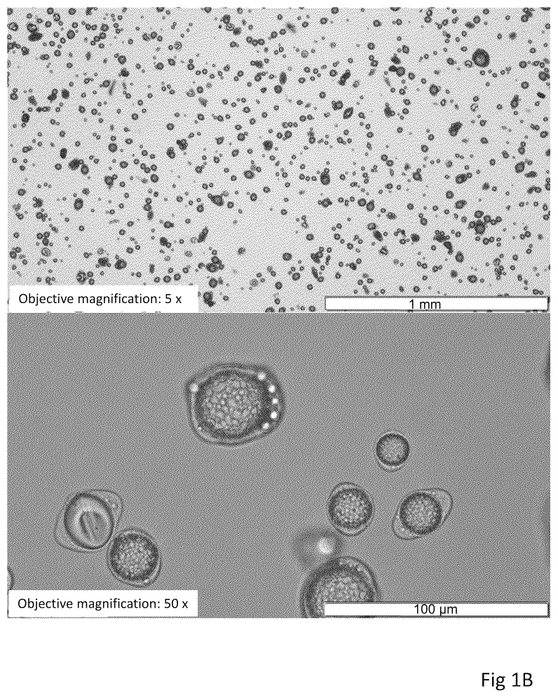

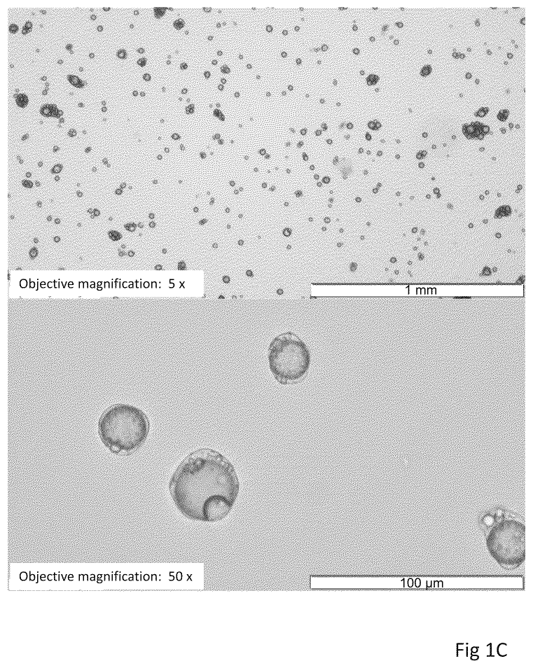

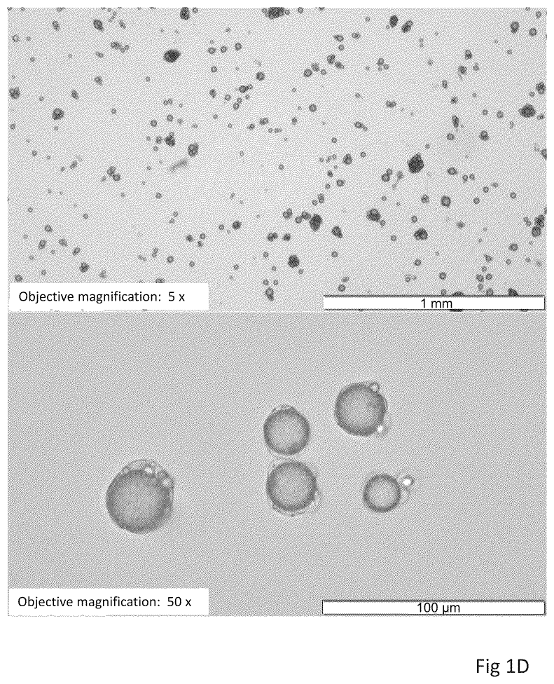

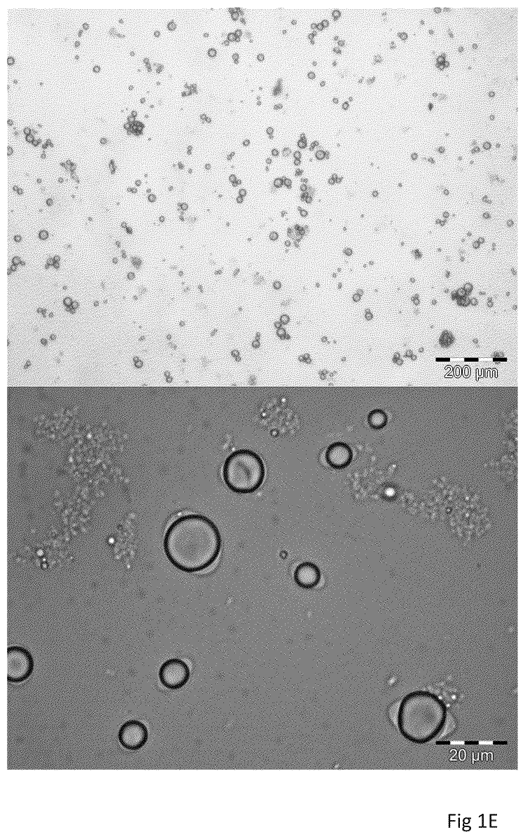

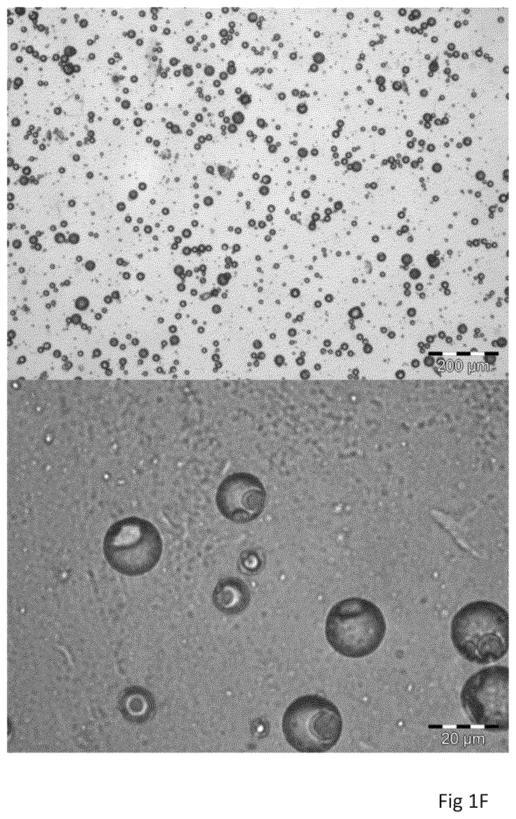

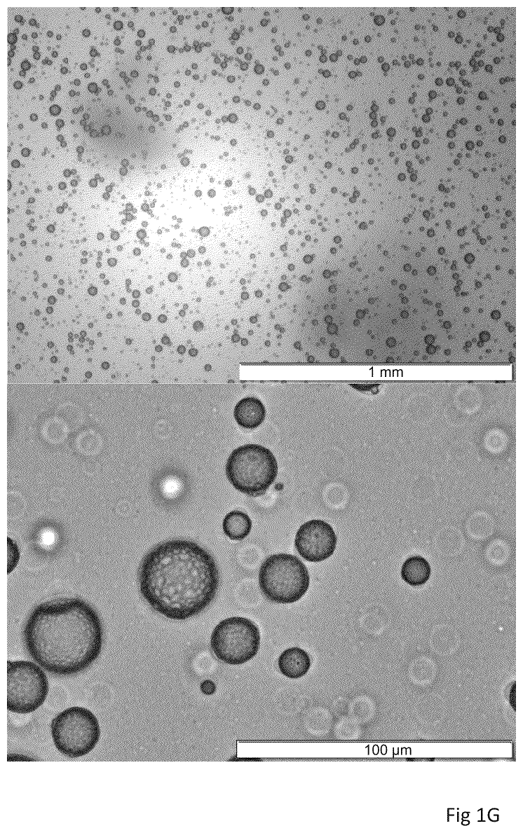

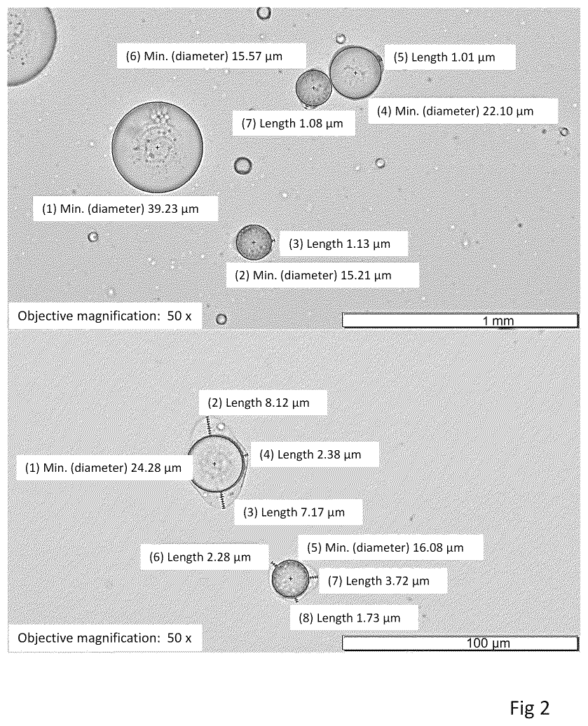

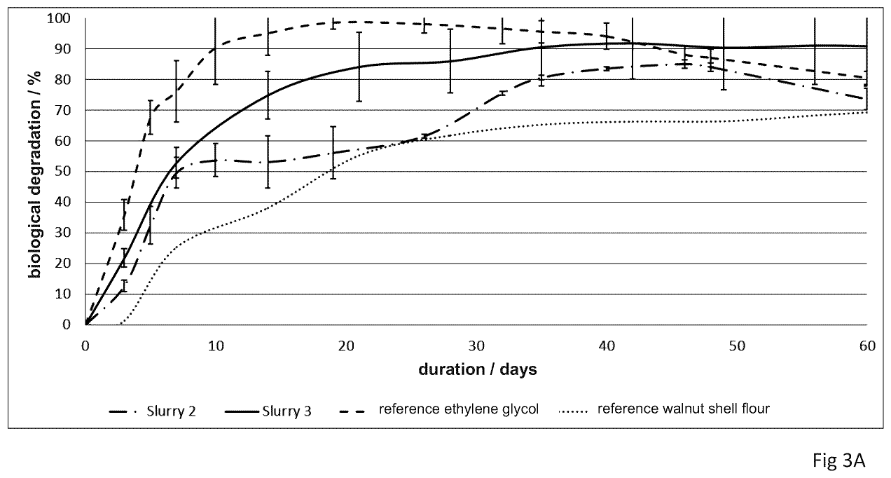

Exemplary light microscope images of the performed layer thickness measurement for Slurry 3 and a comparison of the microcapsule MK1 are depicted in FIG. 2. The result of the measurement of 10 capsules of Slurry 3 is presented in Table 11.

This result for Slurry 3 was confirmed by laser diffraction and through the difference in the modal value of the particle size distribution compared to a microcapsule without the stability layer.

### Example 5-Stability Measurement of the Microcapsules

### 5.1 Preliminary Remarks

To determine the stability of microcapsules, they were stored for up to 4 weeks in a model fabric softener formulation at 40° C., and the concentration of fragrances diffusing from the inside of the capsule into the surrounding formulation was determined using HS-GC/MS. Based on the measurements, the residual amount of perfume oil still in the capsule was calculated.

Model fabric softener formulation based on Rewoquat WE 18 E US from Evonik, following the recipe from the corresponding product data sheet:

For the preparation of the fabric softener base, 94 g of water was heated to 50° C. and 5.65 g of Rewoquat WE 18 E US was stirred into the warmed water. The mixture was cooled to room temperature, and then the microcapsule dispersion was added.

### 5.2 Experimental Procedure

For this purpose, the microcapsule dispersions Slurry 2, Slurry 3, Slurry 5, Slurry 6, as well as MK1, MK2, and MK4 were carefully homogenized and stored with a concentration of 1 wt-% in the model formulation at 40° C., air-tight, in a heating cabinet. As a comparison, the non-encapsulated fragrance with an analogous fragrance concentration in the model formulation was used.

After the predetermined storage duration, the samples were removed from the heating cabinet and an aliquot was weighed into a 20 ml headspace vial. The vial was then immediately sealed.

These samples were analyzed by headspace-SPME (solid-phase micro-extraction) using capillary gas chromatography, and, after detection with a mass-selective detector (MSD), evaluated.

### 5.3 Result

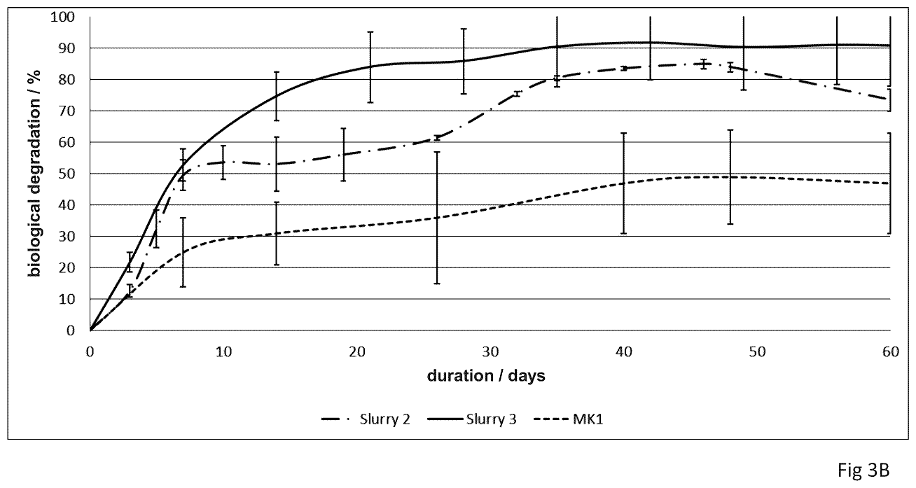

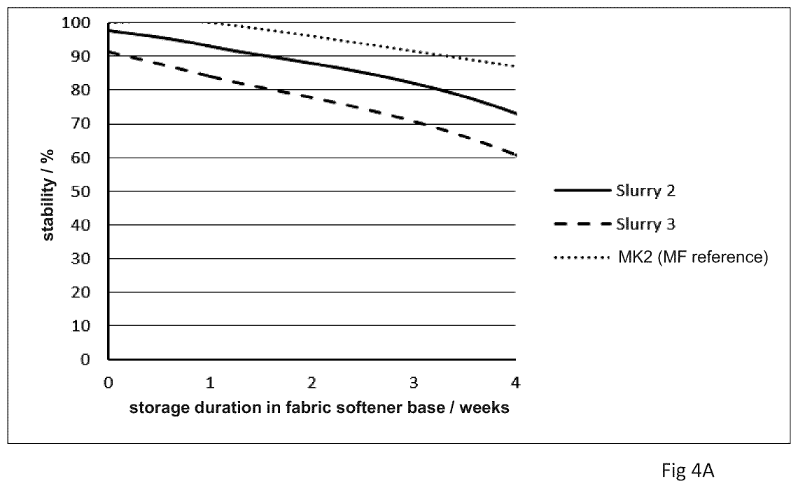

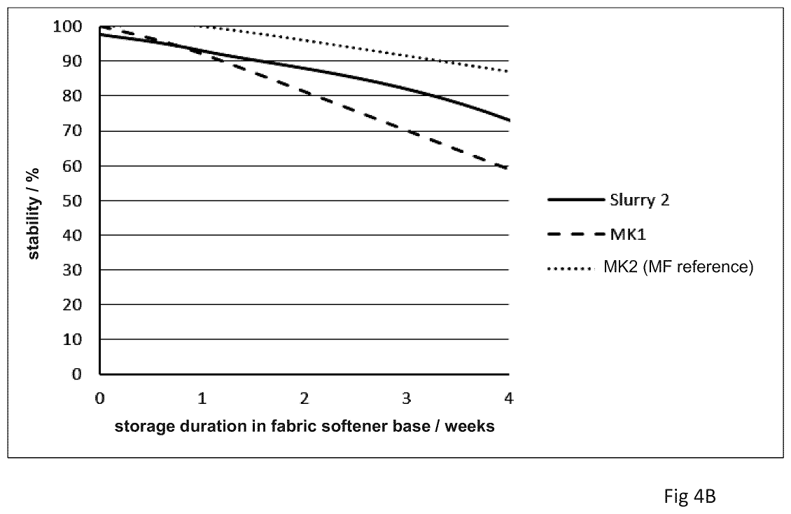

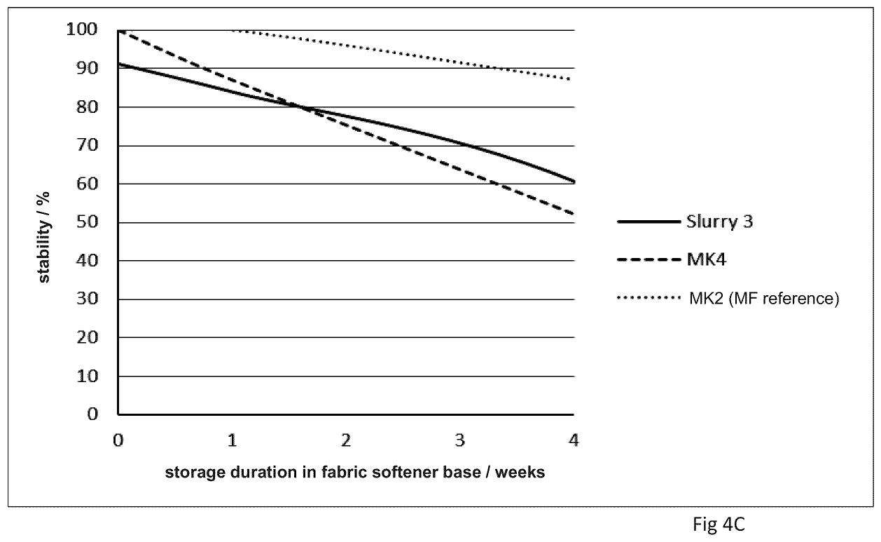

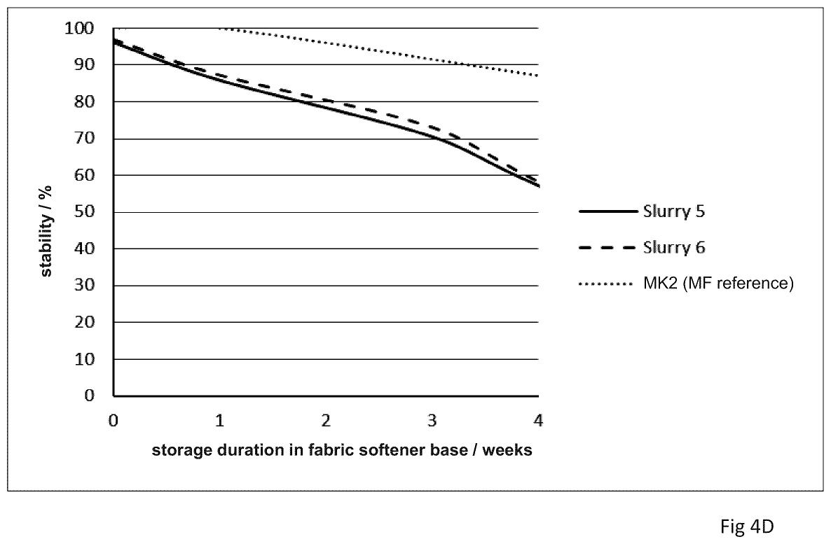

The stability profile of the microcapsule dispersions Slurry 2 and 3, as well as the reference microcapsule dispersion MK2 over 4 weeks is shown in Table 12 and FIG. 4A.

TABLE 12
Measurement values of stability investigation
1: Slurry 2, Slurry 3, and MK2

| Duration/ | MK2 | Slurry 2 | Slurry 3 |
|---|---|---|---|
| weeks | Stability/% | Stability/% | Stability/% |
| 0 | 100 | 98 | 91 |
| 1 | 100 | 93 | 84 |
| 4 | 87 | 73 | 61 |

The stability profile of the microcapsule dispersions Slurry 5 and 6, as well as the reference microcapsule dispersion MK2 over 4 weeks is shown in Table 13 and FIG. 4D.

TABLE 13
Measurement values of stability investigation
1: Slurry 5, Slurry 6, and MK2

| Duration/ | MK2 | Slurry 5 | Slurry 6 |
|---|---|---|---|
| weeks | Stability/% | Stability/% | Stability/% |
| 0 | 100 | 96 | 97 |
| 1 | 100 | 86 | 87 |
| 4 | 87 | 57 | 58 |

As can be inferred from Tables 12 and 13, the microcapsules according to the invention, Slurry 2, Slurry 3, Slurry 5, and Slurry 6, show a stability comparable to the MF-reference MK2 after 4 weeks of storage in a model formulation. Furthermore, it is evident that the microcapsules according to the invention with increased layer thickness of the stability layer, Slurry 2 and 3, exhibit improved stability over a storage duration of 4 weeks compared to the reference capsules MK1 and MK4 (see FIGS. 4B and 4C).

For the calculation of capsule stability, a concentration change of 16 individual constituents of the encapsulated fragrance was considered. A decrease in stability results in the release of the encapsulated fragrance, which can then be detected using headspace-SPME gas chromatography. Since all capsule dispersions were adjusted to a defined oil content of 15 wt. %, a direct comparison of the studied capsule samples is possible. Individual constituents (or their gas chromatographic individual signals), which showed higher concentrations due to measurement-related fluctuations than theoretically possible compared to the reference standard, were only considered up to the theoretical maximum concentration in the evaluation.

### Beispiel 6-Biodegradability Measurement of the Microcapsules (According to OECD 301 F)

### 6.1 General

This experiment is designed to assess the rapid biological degradability of the microcapsules.

The test concentration of the samples to be examined is standardly 1000 mg/l O₂. The sensors and the controller measure the oxygen consumption in a closed system.

The consumption of oxygen and the simultaneous binding of the resulting carbon dioxide to sodium hydroxide pellets create a vacuum in the system. The sensors register and store this pressure over the set measurement period. The stored values are read into the controller via infrared transmission. They can be transferred to a PC and evaluated using the Achat OC program.

To eliminate the influence of the core material on the degradation, perfluorooctane was encapsulated (degradation rate=<1%).

### 6.2 Instruments and Chemicals

Instruments: OxiTop-Control measurement system, WTW, including Controller
OxiTop OC 110 with interface cable for PC, 6 measuring heads
OxiTop C, 6 glass bottles with a total volume of 510 ml each, 6 magnetic stirrers, 6 rubber sheaths, 1 magnetic stir system, and
Achat OC readout software
Drying cabinet ORI-BSB, set to 20° C.
Airstones Oxygenius, 30×15×15 mm3
Aquarium air pump Thomas-ASF No. 1230053
Filter funnel D=90 mm
Suction bottle 2 I
White band filter MN 640 d, D=90 mm, Macherey+Nagel
Manual “System Oxi Top Control”, WTW

Chemicals: Activated sludge from the company's own or a municipal wastewater treatment plant
Ethylene glycol for analysis, Merck
Reference sample with COD (Chemical Oxygen Demand) 1000 mg/l O₂
Walnut shell flour, Senger Naturrohstoffe
Nutrient salt solution from the company's own or a municipal wastewater treatment plant
Sodium hydroxide pellets for analysis >99%, Merck
Cuvette test COD LCK 514, Dr. Lange

### 6.3 Experimental Procedure

### 6.3.1 Preparation of the Microcapsule Slurries

The microcapsule dispersions Slurry 2 and Slurry 3 were prepared according to the descriptions in Examples 2 and 3, with the difference being that the completely persistent perfluorooctane (degradation rate <1%) was used as the core material instead of the perfume oil. This eliminates any potential influence of the core material on the experimental results.

### 6.3.2 Sample Preparation

In the case of extended degradation tests over 60 days, the microcapsule slurries were washed after production by centrifuging three times and redispersing in water to remove dissolved residues. For this, a sample of 20-30 mL is centrifuged for 10 minutes at 12,000 rpm. After the clear supernatant is aspirated, it is refilled with 20-30 ml of water and the sediment is redispersed by shaking.

### 6.3.3 Preparation of the Reference Sample

711.6 mg of ethylene glycol was dissolved in a 1-liter volumetric flask and filled up to the mark. This corresponds to a COD (Chemical Oxygen Demand) of 1000 mg/l O₂. Ethylene glycol is considered to be readily biodegradable and is used here as a reference.

Due to the rapid degradation of ethylene glycol, walnut shell flour was added as an additional reference for the extended 60-day test. Walnut shell flour consists of a mixture of biopolymers, particularly cellulose and lignin, and serves as a bio-based solid reference. Due to the slow degradation of walnut shell flour, the test course can be followed over the full 60-day period. For this, 117.36 g of walnut shell flour was homogeneously dispersed in 11 of water under stirring. Aliquot parts of this mixture were taken under stirring for COD determination. Based on the average COD value of 1290±33 mg/l O₂, the required amount to be used was calculated and transferred into the OxiTop bottles under stirring.

### 6.3.4 Preparation of the Activated Sludge

Activated sludge was collected from the outlet of the aeration tank of either the company's own or a municipal wastewater treatment plant using a 20-liter bucket. After settling for 30 minutes, the supernatant water was discarded.

Subsequently, the concentrated activated sludge in the bucket was continuously aerated for 3 days using the aquarium pump and an airstone.

### 6.3.5 Dry Content Determination of the Activated Sludge

After 3 days, 100 ml of the concentrated activated sludge was filtered through a filter funnel over a white band filter. The filter cake is dried for 24 hours at 105° C. in the drying cabinet.

$$T G = \frac{A}{E}$$

TG=Dry Content of the Activated Sludge in %
E=Initial Weight of the Filter Cake in g
A=Final Weight of the Filter Cake in g
C=TG×10
C=Concentration of the Activated Sludge in g/l
   6.3.6 Adjusting the Samples to a COD of 1000 mg/l O₂

The COD value of the samples to be tested was determined using the cuvette test COD LCK 514. The sample is diluted with water until a COD value of 1000 mg/l O₂ is reached.

### 6.3.7 Preparation of the Test Mixtures

For one sample, 6 OxiTop bottles were used as duplicate determinations were carried out.

The following measurements were conducted in 2 bottles each (duplicate determination):
Biological degradability of the sample
Biological degradability of the ethylene glycol solution (=reference solution)
Blank sample (=determination of the residual degradation of the sludge itself)

Each bottle requires:
25 ml of the sample with a COD of 1000 mg/l 02 (for blank sample, 25 ml of distilled water)
3.5 ml of nutrient solution
44.5 mg of oven-dry sludge
Distilled water to fill up to a total volume of 250 ml

Three sodium hydroxide pellets were placed in each rubber sheath with a spatula. After the bottles were equipped with the sample, nutrient solution, activated sludge, and distilled water, a magnetic stirring bar was placed in each bottle. Then, the rubber sheaths were fitted onto the respective bottle necks and the measuring heads were tightly screwed onto the bottles.

### 6.4 Measurement and Evaluation

The programming of the OxiTop-C measuring heads and the evaluation of the data are described in detail in the “System OxiTop Control” manual by WTW.

### 6.5 Result

The degradation values of the microcapsules measured at different times are presented in Table 14.

TABLE 14
Representation of degradation values pursant to OECD 301 (60 days)

| | Slurry 2 | | Slurry 3 | | Ethylene glycol | | Walnut shell |
|---|---|---|---|---|---|---|---|
| | Degradation [%] | | Degradation [%] | | Degradation [%] | | flour |
| Day | Mean | SD | Mean | SD | Mean | SD | Degradation [%] |
| 0 | 0 | 0 | 0 | 0 | 0 | 0 | 0 |
| 3 | 13 | 2 | 22 | 3 | 36 | 5 | 1 |
| 5 | 32 | 6 | | | 68 | 6 | |
| 7 | 50 | 5 | 53 | 5 | 76 | 10 | 25 |
| 10 | 54 | 5 | | | 90 | 12 | |
| 14 | 53 | 9 | 75 | 8 | 95 | 7 | 38 |
| 19 | 56 | 8 | | | 99 | 2 | |
| 21 | | | 84 | 11 | | | 55 |
| 26 | 62 | 1 | | | 98 | 3 | |
| 28 | | | 86 | 10 | | | 62 |
| 32 | 76 | 1 | | | 97 | 5 | |
| 35 | 81 | 1 | 91 | 13 | 96 | 4 | 65 |
| 40 | 84 | 1 | | | 94 | 4 | |
| 42 | | | 92 | 12 | | | 66 |
| 46 | 85 | 1 | | | 88 | 3 | |
| 48 | 84 | 1 | | | 87 | 3 | |
| 49 | | | 90 | 14 | | | 66 |
| 56 | | | 91 | 13 | | | 68 |
| 60 | 74 | 3 | 91 | 13 | 81 | 2 | 69 |

The microcapsule dispersions Slurry 2 and Slurry 3 demonstrate very good biodegradability in the OECD301F test. After 14 days (Slurry 3) and 26 days (Slurry 2), they meet the requirements of the OECD/ECHA, as they show a degradation degree of >60%.

The degradation profile of the reference sample ethylene glycol indicates a healthy inoculum and also demonstrates the instrumental functionality throughout the entire duration of the experiment.

The walnut shell flour is characterized by the typical, stepwise degradation profile expected for a complex mixture of biopolymers. The continuous increase in biological degradability over the entire 60-day experimental period also suggests a healthy inoculum.

### Beispiel 7-Determination of the Coloration of Microcapsule Dispersions

### 7.1 Experimental Procedure

The color of the microcapsule dispersions Slurry 2 and 3 as color coordinate in the L*a*b* color space was determined using the following experimental procedure.

To determine the color of microcapsule dispersions, the portable spectrophotometer “spectro-color d/8° C.” from Dr. Lange was used, in conjunction with glass cuvettes for liquids. Furthermore, the measurement took place in the associated measurement setup, which darkens the sample during measurement (thus minimizing the influence of scattered light). Before starting the measurements, a calibration was performed against a black and white standard (LZM268 standard set).

The respective capsule dispersion is filled undiluted into a round glass cuvette (about 5-6 mL). Using the associated PTFE plunger, the measurement area is adjusted to a defined layer thickness, and any air inclusions in the dispersion are removed.

The color measurement was carried out as a triple determination, with the cuvette being rotated by about 30° after each individual measurement. Subsequently, the average value and standard deviation are calculated.

### 7.2 Result

TABLE 15
Determination of the color coordinate of Slurry 2 and 3 in
the L*a*b* color space after production and after storage

| | Maturation | | | | | | |
|---|---|---|---|---|---|---|---|
| | time / days | Raw data | | | | Mean | SD |
| Slurry | 0 | L | 51.76 | 52.13 | 52.17 | 52 | 0 |
| 3 | | a | 10.94 | 10.79 | 10.65 | 11 | 0 |
| | | b | 11.67 | 10.62 | 10.68 | 11 | 0 |
| | 48 | L | 51.09 | 51.25 | 51.17 | 51 | 0 |
| | | a | 13.05 | 13.02 | 13.03 | 13 | 0 |
| | | b | 14.04 | 13.39 | 13.49 | 14 | 0 |
| Slurry | 0 | L | 64.82 | 64.78 | 64.79 | 65 | 0 |
| 2 | | a | 3.86 | 3.74 | 3.61 | 4 | 0 |
| | | b | 12.62 | 12.99 | 13.16 | 13 | 0 |
| | 48 | L | 63.82 | 63.83 | 63.76 | 64 | 0 |
| | | a | 4.35 | 4.4 | 4.38 | 4 | 0 |
| | | b | 14.27 | 13.88 | 14.23 | 14 | 0 |

### Example 8-Influence of Heat Treatment on Color Stability

To determine the influence of heat treatment in the final curing step on color stability, the manufacturing process of Slurry 3 was modified. In this modified process, the heating of the reaction mixture to 40° C. for 1 hour before stirring for 14 hours at room temperature was omitted. The resulting microcapsule dispersion is referred to as Slurry 3A.

Slurries 3 and 3A were prepared in parallel, and immediately after production, the color coordinates of samples from both microcapsule dispersions were determined according to the protocol described in Example 7. On days 1, 2, 3, 4, and 8, samples of Slurries 3 and 3A were taken, and once again, the color coordinates of these samples were determined. Three samples per microcapsule dispersion and day were measured, and the average for the three L*a*b* positions was calculated.

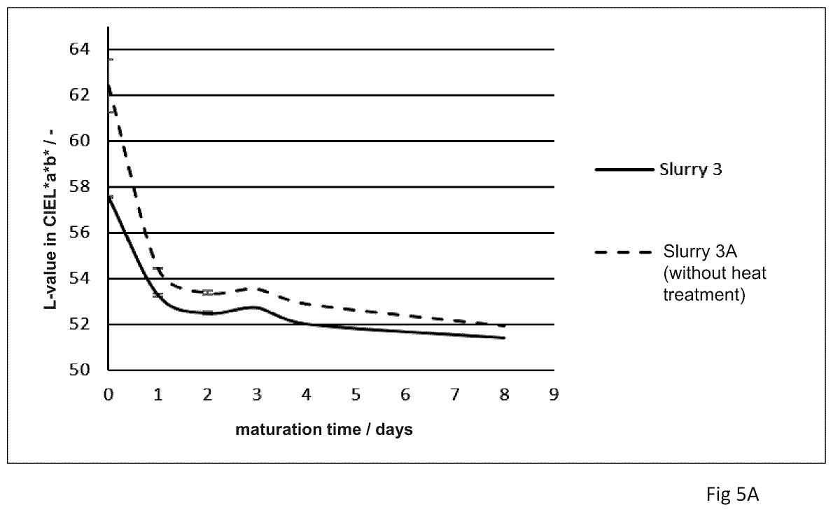

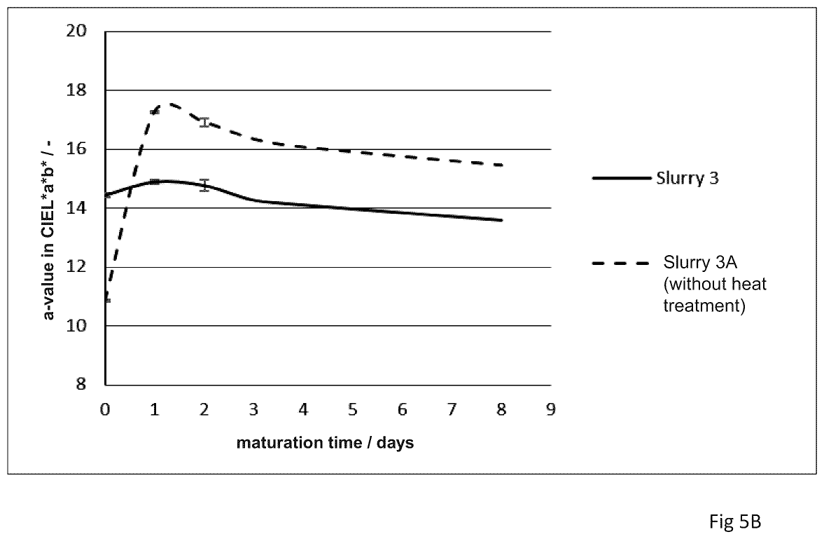

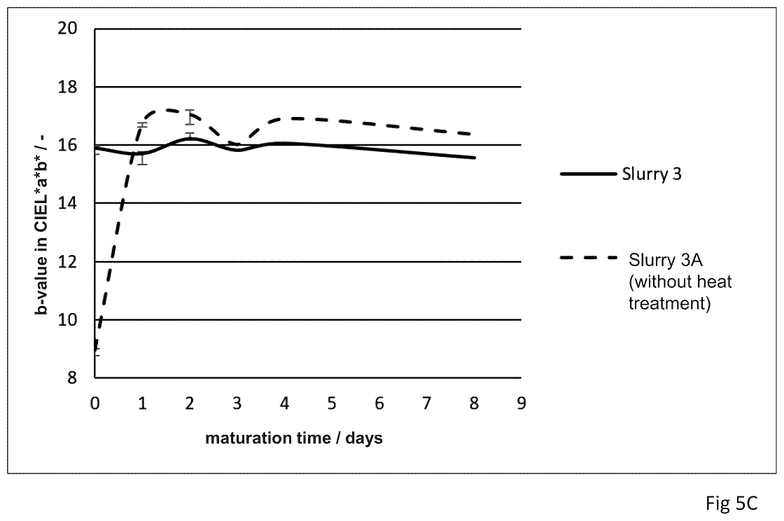

FIG. 5 shows the temporal development of the L*a*b* positions for both microcapsule dispersions, Slurry 3 and 3A. The diagrams clearly indicate that the heat-treated microcapsule dispersion Slurry 3 exhibits constant color stability from the outset. In contrast, the non-heat-treated microcapsule dispersion shows a more significant color change, particularly at the beginning of the storage period, with values of ΔL* of approximately 10, Aa* of approximately 5, and Ab* of approximately 8.

Regarding further advantageous embodiments of the device according to the invention, reference is made to the general part of the description and the attached claims to avoid repetition.

Finally, it is expressly noted that the exemplary embodiments of the means and uses according to the invention described above serve only to explain the claimed subject matter and do not limit it to the embodiments described.

1. Biodegradable microcapsules, comprising a core material and a shell, wherein the shell consists of at least one barrier layer and at least one stability layer, wherein the barrier layer surrounds the core material, wherein the stability layer comprises at least one biopolymer, and is arranged on the outer surface of the barrier layer, and wherein an emulsion stabilizer is arranged at the transition from barrier layer to stability layer.

2. The biodegradable microcapsules according to claim 1, wherein in the production of the microcapsules, the surface of the barrier layer is brought into contact with the emulsion stabilizer before the formation of the stability layer, whereby the capacity of the surface for structural attachment of the stability layer is increased.

3. The biodegradable microcapsules according to claim 1, wherein the emulsion stabilizer is a polymer or copolymer, consisting of one or more monomers selected from:
(1) Acrylic acid derivatives of the general formula (I) 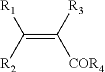
wherein
R₁, R₂ and R₃ are selected from hydrogen and a C₁₋₄-alkyl group, wherein R₁ and R₂ are particularly hydrogen and R₃ is particularly hydrogen or methyl; and
R₄ is for —OX or —NR₅R₆, wherein X is hydrogen, an alkali metal, an ammonium group, or a C₁-C₁₈ alkyl possibly substituted by —SO₃M or —OH, wherein M is hydrogen, an alkali metal, or ammonium, wherein the optionally —SO₃M or —OH substituted C₁-C₁₈ alkyl is preferably methyl, ethyl, n-butyl, 2-ethylhexyl, 2-sulfoethyl, or 3-sulfopropyl, wherein R₅ and R₆ are independently selected from hydrogen or a C₁-C₁₀ alkyl possibly substituted by —SO₃M, wherein at least one of R₅ and R₆ is not hydrogen, and wherein preferably R₅ is H and R₆ is 2-methyl-propan-2-yl-1-sulfonic acid;
(2) N-vinylpyrrolidone; and
(3) styrene;
wherein the emulsion stabilizer is preferably an acrylate copolymer containing 2-acrylamido-2-methylpropanesulfonic acid (AMPS) and is particularly preferably available under the trade name Dimension PA 140.

4. The biodegradable microcapsules according to claim 1, wherein the proportion of the emulsion stabilizer relative to the total weight of the capsule is in the range of 0.5 to 15.0 wt. %, preferably in the range of 1 to 11 wt. %, more preferably in the range of 2 to 7 wt. %.

5. The biodegradable microcapsules according to claim 1, wherein the barrier layer is composed of one or more components selected from the group consisting of an aldehydic component, an aromatic alcohol, an amine component, an acrylate component, and an isocyanate component.

6. The biodegradable microcapsules according to claim 5, the barrier layer contains an aldehydic component, selected from the group consisting of formaldehyde, glutaraldehyde, succinaldehyde, furfural, and glyoxal, and preferably the proportion of the aldehydic component for the polycondensation relative to the total weight of the second shell is in the range of 5 to 50 wt. %, preferably in the range of 10 to 30 wt. %, more preferably in the range of 15 to 20 wt. %.

7. The biodegradable microcapsules according to claim 5, wherein the barrier layer contains an aromatic alcohol, selected from the group consisting of resorcinol, phloroglucin, and aminophenol, and preferably the proportion of the aromatic alcohol relative to the total weight of the barrier layer is in the range of 1.0 to 20 wt. %, preferably in the range of 6 to 16 wt. %, more preferably in the range of 10 to 14 wt. %.

8. The biodegradable microcapsules according to claim 1, wherein the barrier layer contains an amine component, selected from the group consisting of melamine, melamine derivatives, urea, and combinations thereof, and preferably the proportion of the amine component relative to the total weight of the barrier layer is in the range of 20 wt. % to 85 wt. %, preferably in the range of 40 wt. % to 80 wt. %, more preferably in the range of 55 wt. % to 70 wt. %

9. The biodegradable microcapsules according to claim 1, the biopolymers of the stability layer are selected from the group consisting of proteins such as gelatin, whey protein, plant storage protein; polysaccharides such as alginate, gum arabic, modified gum, chitin, dextran, dextrin, pectin, cellulose, modified cellulose, hemicellulose, starch or modified starch; phenolic macromolecules such as lignin; polyglucosamines such as chitosan, polyvinyl esters, such as polyvinyl alcohols and polyvinyl acetate; phosphazenes, and polyesters such as polylactide or polyhydroxyalkanoate, wherein the biopolymers are particularly gelatin and/or alginate.

10. The biodegradable microcapsules according to claim 1, wherein the biopolymer in the stability layer is crosslinked with a curing agent and the curing agent of the stability layer is selected from the group consisting of an aldehyde, such as glutaraldehyde, formaldehyde or glyoxal, a tannin, an enzyme such as transglutaminase, and an organic anhydride like maleic anhydride, an epoxy compound, a polyvalent metal cation, an amine, a polyphenol, a maleimide, a sulfide, a phenol oxidase, a hydrazide, an isocyanate, an isothiocyanate, an N-hydroxysulfosuccinimide derivative, a carbodiimide derivative, and a polyol, wherein the curing agent is particularly preferably glutaraldehyde or glyoxal.

11. The biodegradable microcapsules according to claim 1, wherein the proportion of the barrier layer in the shell relative to the total weight of the shell is at most 30 wt. %, preferably at most 25 wt. %, and more preferably at most 20 wt. %.

12. The biodegradable microcapsules according to claim 1, wherein the stability layer has an average thickness of at least 1 μm, preferably at least 2 μm, and more preferably at least 3 μm.

13. The biodegradable microcapsules according to claim 1, wherein the microcapsule has a third layer that is arranged on the outside of the stability layer and contains a component selected from amines, organic salts, inorganic salts, alcohols, ethers, polyphosphazenes, and noble metals, wherein the proportion of the third layer in the shell relative to the total weight of the shell is at most 35%, preferably at most 30 wt. %, and more preferably at most 25 wt. %.

14. The biodegradable microcapsules according to claim 1, wherein the core material is selected from the group consisting of fragrances, flavors, phase change materials, colorant solutions, lubricants and oils, silicone oils, cosmetic active ingredients, pharmaceutical active ingredients, catalysts, self-healing reagents, initiator systems, adhesive components, and reactive components.

15. The biodegradable microcapsules according to claim 1, wherein the biodegradable microcapsules have at least one, preferably all of the following characteristics:
a biodegradability of the shell measured according to OECD 301 F of at least 40%, preferably at least 50%, more preferably at least 60%, and particularly 70%; and
an impermeability that ensures the release of no more than 50 wt. % of the used core material after storage over a period of 4 weeks at a temperature of 0 to 40° C., preferably no more than 45 wt. %, and more preferably no more than 30 wt. %

16. A microcapsule dispersion, containing biodegradable microcapsules according to claim 1, characterized in that they have a color coordinate in the L*a*b* color space with an L* value of at least 50, and preferably the color coordinate of the microcapsule dispersion in the L*a*b* color space has an L* value of at least 50 after a period of at least four weeks.

17. A product containing biodegradable microcapsules according to claim 1, wherein the product is selected from the group consisting of an adhesive system; a pharmaceutical product; a coating material, in particular a coated paper; a heat storage coating, a self-healing coating, or a corrosion coating; and coatings containing such microcapsules for functional packaging materials.

18. Use of the biodegradable microcapsules according to claim 1 for the production of a product, wherein the product is not a laundry and cleaning agent and/or a cosmetic product.

19. Use of an emulsion stabilizer to increase the amount of a stability layer that can be deposited on the surface of a barrier layer, wherein the barrier layer and the stability layer form the capsule wall of a microcapsule, wherein the emulsion stabilizer is a polymer or copolymer, consisting of one or more monomers selected from
(1) Acrylic acid derivatives of the general formula (I) 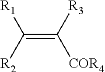
wherein
R₁, R₂ and R₃ are selected from hydrogen and a C₁₋₄-alkyl group, wherein R₁ and R₂ are particularly hydrogen and R₃ is particularly hydrogen or methyl; and
R₄ is for —OX or —NR₅R₆, wherein X is hydrogen, an alkali metal, an ammonium group, or a C₁-C₁₈ alkyl possibly substituted by —SO₃M or —OH, wherein M is hydrogen, an alkali metal, or ammonium, wherein the optionally-SO₃M or —OH substituted C₁-C₁₈ alkyl is preferably methyl, ethyl, n-butyl, 2-ethylhexyl, 2-sulfoethyl, or 3-sulfopropyl, wherein R₅ and R₆ are independently selected from hydrogen or a C₁-C₁₀ alkyl possibly substituted by —SO₃M, wherein at least one of R₅ and R₆ is not hydrogen, and wherein preferably R₅ is H and R₆ is 2-methyl-propan-2-yl-1-sulfonic acid;
(2) N-vinylpyrrolidone; and
(3) styrene;
wherein the emulsion stabilizer is preferably an acrylate copolymer containing 2-acrylamido-2-methylpropanesulfonic acid (AMPS) and is particularly preferably available under the trade name Dimension PA 140; and
wherein the barrier layer is composed of one or more components selected from the group consisting of an aldehydic component, an aromatic alcohol, an amine component, an acrylate component, and an isocyanate component, and the stability layer comprises at least one biopolymer.

20. Use of the polyacrylic acid derivative according to claim 19, wherein the barrier layer contains an aldehydic component, selected from the group consisting of formaldehyde, glutaraldehyde, succinaldehyde, furfural, and glyoxal, and preferably the proportion of the aldehydic component for the polycondensation relative to the total weight of the second shell is in the range of 5 to 50 wt. %, preferably in the range of 10 to 30 wt. %, more preferably in the range of 15 to 20 wt. % and the biopolymers of the stability layer are selected from the group consisting of proteins such as gelatin, whey protein, plant storage protein; polysaccharides such as alginate, gum arabic, modified gum, chitin, dextran, dextrin, pectin, cellulose, modified cellulose, hemicellulose, starch or modified starch; phenolic macromolecules such as lignin; polyglucosamines such as chitosan, polyvinyl esters, such as polyvinyl alcohols and polyvinyl acetate; phosphazenes, and polyesters such as polylactide or polyhydroxyalkanoate, wherein the biopolymers are particularly gelatin and/or alginate.

21. A method for producing biodegradable microcapsules according to claim 1, characterized by the following steps:
a) preparing an oil-in-water emulsion by emulsifying a core material in an aqueous phase in the presence of the wall-forming component(s) of the inner barrier layer with the addition of protective colloids;
b) deposition and curing of the wall-forming component(s) of the barrier layer, wherein the wall-forming component(s) of the barrier layer preferably are an aldehydic component, an amine component, and an aromatic alcohol, particularly preferably formaldehyde, melamine, and resorcin;
c) addition of an emulsion stabilizer;
d) addition of the wall-forming component(s) of the stability layer, followed by deposition and curing, wherein the wall-forming components of the stability layer are at least one biopolymer, preferably a protein and/or a polysaccharide, particularly preferably gelatin and alginate, as well as a curing agent, preferably glutaraldehyde or glyoxal; and
e) optionally, the addition of the wall-forming component(s) of the outer, third shell layer, followed by deposition and curing, wherein the wall-forming component(s) of the outer, third shell layer preferably is an amine component, particularly melamine.

22. The method according to claim 21, characterized in that the emulsion stabilizer is a polymer or copolymer, consisting of one or more monomers selected from:
(1) Acrylic acid derivatives of the general formula (I) 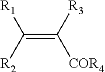
wherein
R₁, R₂ and R₃ are selected from hydrogen and a C₁₋₄-alkyl group, wherein R₁ and R₂ are particularly hydrogen and R₃ is particularly hydrogen or methyl; and
R₄ is for —OX or —NR₅R₆, wherein X is hydrogen, an alkali metal, an ammonium group, or a C₁-C₁₈ alkyl possibly substituted by —SO₃M or —OH, wherein M is hydrogen, an alkali metal, or ammonium, wherein the optionally-SO₃M or —OH substituted C₁-C₁₈ alkyl is preferably methyl, ethyl, n-butyl, 2-ethylhexyl, 2-sulfoethyl, or 3-sulfopropyl, wherein R₅ and R₆ are independently selected from hydrogen or a C₁-C₁₀ alkyl possibly substituted by —SO₃M, wherein at least one of R₅ and R₆ is not hydrogen, and wherein preferably R₅ is H and R₆ is 2-methyl-propan-2-yl-1-sulfonic acid;
(2) N-vinylpyrrolidone; and
(3) styrene;
wherein the emulsion stabilizer is preferably an acrylate copolymer containing 2-acrylamido-2-methylpropanesulfonic acid (AMPS) and is particularly preferably available under the trade name Dimension PA 140.

23. The method according to claim 21, characterized in that during the curing step e) it is heated to a temperature in the range of 20° C. to 80° C., preferably in the range of 30° C. to 60° C., more preferably in the range of 40° C. to 50° C.
